SEQUENCE LISTING (16 sequences)

SEQ ID NO: 1 - protein, 9 aa, Spinacia oleracea
  REGION 1..9: note = motif
MAEIGYSVC

SEQ ID NO: 2 - protein, 6 aa, Spinacia oleracea
  REGION 1..6: note = motif
KWMCLR

SEQ ID NO: 3 - protein, 8 aa, Spinacia oleracea
  REGION 1..8: note = motif
HVGCVVDR

SEQ ID NO: 4 - DNA, 20 bp, Spinacia oleracea
  misc_feature 1..20: note = Forward primer LRR domain (Alpha)
  source 1..20: mol_type = genomic DNA
acaagtggatgtgtcttagg

SEQ ID NO: 5 - DNA, 19 bp, Spinacia oleracea
  misc_feature 1..19: note = Reverse primer LRR domain (Alpha)
  source 1..19: mol_type = genomic DNA
ttcgccctcatcttcctgg

SEQ ID NO: 6 - DNA, 18 bp, Spinacia oleracea
  misc_feature 1..18: note = Forward primer LRR domain (Beta)
  source 1..18: mol_type = genomic DNA
tcacgtgggttgtgttgt

SEQ ID NO: 7 - DNA, 1597 bp, Spinacia oleracea
  misc_feature 1..1597: note = Amplicon of LRR domain of the beta-WOLF 0 allele (Viroflay)
  source 1..1597: mol_type = genomic DNA
tcacgtgggttgtgttgtcgatagagatccagaaatagtctttttatgtagcaataagattcgttcgtatattagcggtcgctgcataaagaatccggtggattcacaaatagacaactggatgtgccttagggtgttggacttgtcagattcatgtgttaaagatttgtctgattcaataggtaagctgctgcacttaaggtatcttaacctctcttctaatataaagttggagataatccctgatgcaattacaagactgcataacttgcagacactacttttagaagattgcagaagtttaaaggagttgccaaaagatttttgcaaattggtcaaactgaggcacttggaattacagggttgtcatgatttgattggtatgtcatttggaatggataagctaactagtcttagaatactaccaaacattgtggtgggtaggaaggaacaaagtgttgatgatgagctgaaagccctaaaaggcctcaccgagataaaaggctccattgatatcacaatctattcaaaatatagaagagttgaaggcatgaatggcacaggaggaggagctgggtatttgaagagcatgaaacatctcacgggggttaatattacatttgatgaaggtggatgtgttaaccctgaagctgtgtatttgaagagcatgaaacatctcacgagggttattattatatttgattataaaggtggatgtgttaaccctgaagctgtgttggcaaccctagagccaccttcaaatatcaagaggttagagatgtggcattacagtggtacaacaattccagtatggggaagagcagagattaattgggcaatctccctctcacatcttgtcgacatcacgcttgaagattgttacaatttgcaggagatgccagtgctgagtaaactgcctcatttgaaatcactggaacttacagagttggataacttagagtacatggagagtagaagcagcagcagtagcagtgacacagaagcagcaacaccagaattaccaacattcttcccttcccttgaaaaacttacactttggcgtctggacaagttgaagggttttgggaacaggagatcgagtagttttccccgcctctctaaattggaaatctggaaatgtccagatctaacgtcatttccttcttgtccaagccttgaagagttggaattgaaagaaaacaatgaagcgttgcaaataatagtaaaaataacaacaacaagaggtaaagaagaaaaagaagaagacaagaatgctggtgttggaaattcacaagatgatgacaatgtcaaattatggaaggtggaaatagacaatctgggttatctcaaatcactgcccacaaattgtctgactcacctcgaccttacaataagtgattccaaggagggggagggtgaatgggaagttggggatgcatttcagaagtgtgtatcttctttgagaagcctcaccataatcggaaatcacggaataaataaagtgaagagactgtctggaagaacagggttggagcatttcactctgttggaatcactcaaactttcagatatagaagaccaggaagatgagggcgaa

SEQ ID NO: 8 - protein, 532 aa, Spinacia oleracea
  REGION 1..532: note = Amino acid sequence encoded by amplicon of LRR domain Beta Wolf 0(Viroflay)
HVGCVVDRDPEIVFLCSNKIRSYISGRCIKNPVDSQIDNWMCLRVLDLSDSCVKDLSDSIGKLLHLRYLNLSSNIKLEIIPDAITRLHNLQTLLLEDCRSLKELPKDFCKLVKLRHLELQGCHDLIGMSFGMDKLTSLRILPNIVVGRKEQSVDDELKALKGLTEIKGSIDITIYSKYRRVEGMNGTGGGAGYLKSMKHLTGVNITFDEGGCVNPEAVYLKSMKHLTRVIIIFDYKGGCVNPEAVLATLEPPSNIKRLEMWHYSGTTIPVWGRAEINWAISLSHLVDITLEDCYNLQEMPVLSKLPHLKSLELTELDNLEYMESRSSSSSSDTEAATPELPTFFPSLEKLTLWRLDKLKGFGNRRSSSFPRLSKLEIWKCPDLTSFPSCPSLEELELKENNEALQIIVKITTTRGKEEKEEDKNAGVGNSQDDDNVKLWKVEIDNLGYLKSLPTNCLTHLDLTISDSKEGEGEWEVGDAFQKCVSSLRSLTIIGNHGINKVKRLSGRTGLEHFTLLESLKLSDIEDQEDEGE

SEQ ID NO: 9 - DNA, 1392 bp, Spinacia oleracea
  misc_feature 1..1392: note = Amplicon of LRR domain of the alpha-WOLF 27 allele
  source 1..1392: mol_type = genomic DNA
tggatgtgtcttaggatgttggacttgtcaaggccggatgttaaaaatttgcctaattcaataggtaaattgttgcacttgaggtatcttaacctgtcttgtaatgatgatctgttgatactccctgatgcaattacaagactgcataatttgcagacactgcttttaaaagattgcggaagtttaaaggagttgccaaaagatttttgcaaattggtcaaactgagacacttggatttaaggtattgttggcgtttgattggtatgccattgggaatggatatgctaactagtcttagagtactgccatactttgtggtgggtaggaagaaacaaagtgttgatgatgagctgaaagcccttaaaggcctcaccgagataaaaggctccattaatatcaaaatctgtgaaaattatagaatagttgaaggcatgaatgacacaggaggagctgggtatttgaagagcatgaaacatctcacgggggttgatattacatttgatggtggatgtgttaaccctgaagctgtgttggaaaccctagagccaccttcaaatatcaagaggttatctatagataattacgatggtacaacaattccagtatggggaagagcagagattaattgggcaatctccctctcacatcttgtcgacatttggttttgtggttgtagtaatttgcaggagatgccagtgctgagtaaactgcctcatttgaaatcactgaatctttttaagttttgtaagttagagtacatggagagtagaagcagcagcagtagcagtgacacagaagcagcaacaccagaattaccaacattcttcccttcccttgaaaaacttacactttggtatctggaaaagttgaagggtttggggaacaggagatcgagtagttttccccgcctctctgaattggaaatctgggaatgcccagatctaacgtggtttcctccttgtccaagccttaaaacgttgaaattggaaaaaaacaatgaagcgttgcaaataatagtaaaaataacaacaacaagaggtaaagaagaaaaagaagaagacaagaatgctggtgttggaaattcacaagatgatgacaatgtcaaattacggaaggcggaaatagacaatctgggttatctcaaatcactgcccacaaattgtctgactcacctcgacattacaataagagattccaaggagggggagggtgaatgggaagttggggaggcatttcagaagtgtgtatcttctttgagaaagctcagcataatcggaaatcacggaataaataaagtgaagagactgtctggaagaacagggttggagcatttcactctgttggactcactcaaattttcaaagatagaagaccaggaagatgagggcgaa

SEQ ID NO: 10 - protein, 464 aa, Spinacia oleracea
  REGION 1..464: note = Amino acid sequence encoded by amplicon of LRR domain ofalpha-WOLF 27
WMCLRMLDLSRPDVKNLPNSIGKLLHLRYLNLSCNDDLLILPDAITRLHNLQTLLLKDCGSLKELPKDFCKLVKLRHLDLRYCWRLIGMPLGMDMLTSLRVLPYFVVGRKKQSVDDELKALKGLTEIKGSINIKICENYRIVEGMNDTGGAGYLKSMKHLTGVDITFDGGCVNPEAVLETLEPPSNIKRLSIDNYDGTTIPVWGRAEINWAISLSHLVDIWFCGCSNLQEMPVLSKLPHLKSLNLFKFCKLEYMESRSSSSSSDTEAATPELPTFFPSLEKLTLWYLEKLKGLGNRRSSSFPRLSELEIWECPDLTWFPPCPSLKTLKLEKNNEALQIIVKITTTRGKEEKEEDKNAGVGNSQDDDNVKLRKAEIDNLGYLKSLPTNCLTHLDITIRDSKEGEGEWEVGEAFQKCVSSLRKLSIIGNHGINKVKRLSGRTGLEHFTLLDSLKFSKIEDQEDEGE

SEQ ID NO: 11 - DNA, 7978 bp, Spinacia oleracea
  misc_feature 1..7978: note = genomic DNA sequence of the alpha-WOLF 27 allele
  source 1..7978: mol_type = genomic DNA
gttctgttttttatggcacagatatccctcatttgcagctctacttctacaaacatctttcattctttcgttttccttttgattcatgtaacagttgaaccttctttcatgactgatatagaatcaggcagctacttcactacttctatgttgatcttattttgtaataaactttgatagattgaataaaggttgtttgcagtgacttcttaagatgtgattagaagtccataatcactttaaggtagtttttctttacatgattaaggtttttccgaggcctttctattgctttgttggttactgtcatgacatatggtttttctttgcttcttatatcatatggtcctcactcaattttttaatataaagtttctcattggttgactataatacgttatagcaccttataatattttatttaatatacaattttatgtattttacctttttcatattttttcgtgatctaccttctcatatgagctacactaatttggtagctgtttatgcaaatcttgtaccaacggttggctatttgctcaaatttttttttttttttttcgagctagtcattttatgatcattgaagtttgctcttatattatcatttatgtattttaccttttttacatttttttcgtgatctacctgctcatatgagccacactaatttggtagctgcttatacaattcttgtatcaacggttggctacttgttcaaatatttttatttttttacgagtaagtcattttatgatcattgaagttgctctaatattatcatggacctattaacgcatgaataattaactcggtaggaattagtttcaaaataaaattcccctcacaaaaaaaaaaaaaaaaaaaaaaaaaaaaatcagaaaaccaaccttctccagtttactgttgtctaaagccaaagagcatggaattttccagtaatcgcagaccccaaattctcttctccaatcgtccctgtcaatttcagcaattgaatcaatcgttgattttaggatttgccgccaaaaaaatgaaaaatccatgaattttagggttcaaatttgatccgtaattgggaaaattttcagcaattgatcttccaaatcattcatacttgtttccagactgcaaatgaaaggtgcgaactttatactgcattttgattttccattactgtaatttattaagatgaactgcaatttgcaattgttttattcgactactcatctttaaatcaaattgctaaattgctagctaattttcttatcatattgccaaaaatttgttgcttaaatgattccatttctctaattatttttgttttattggtagataaataattaaatatcagccccattaattgaatattcaaaggaaatgtatggtccaaaaatggcgtttaatagtcaatgccgtgttttatggggtggtggagtactatatgactgtgtgtggacttggagaagactagagagtattgattatcaaaatatggaccctgaaaatgaaaatgaaaatgatgtttttacactttaaaatcgtcaagaaacaacaatcctctttagcaatagtatttacacgcgttatttgcacggacttcaatgcaaatagtataaatttacagtcaaagttttcattctaaagcgtaaataactttcatgaatggaggacggtagtataagtataacgttatggcctaccattttcttatcatattcacataaatttgttgctaaaagttgttttacttggctaaaatacttttgttcttattggcagataaacatcagtccattattggccaacttgaacatatacctccaaacaataatcaataatgtcgattatgaagtttgtgaatgcaatttattatcactttcatttataaaatgactacttgattaacacatacaatattacctttctccaaacaccctttcaattctgcttaatcttgttttctcatcatctcttcatctttctgaaaacacaacccaatggccgaaatcggatactcggtttgtgcgaaactcatcgaagtgattggcagtgagctgatcaaagagatttgcgacacatggggttacaaatctcttcttgaggacctcaacaaaactgtattgacggtcaggaacgttctcattcaggccggggtgatgcgggagcttactagtgaacaacaaggtttcattgcagaccttaaagatgttgtttatgatgctgatgacttgttcgacaagttactcactcgtgctgagcgaaaacagattgatggaaacgaaatctctgaaaaggtacgtcgtttcttttcctctagtaacaagatcggtcaagcttactacatgtctcgtaaggttaaggaaattaagaagcagttggatgaaattgttgataggcatacaaaatttgggtttagtgctgagtttatacctgtttgtagggaaagggggaacgagagggaaacacgttcatatatagatgtcaagaatattcttgggagggataaagataagaatgatatcatagataggttgcttaatcgtaatgataatgaagcttgtagtttcctgaccatagtgggagcgggaggattgggaaaaactgctcttgcccaacttgtgttcaatgatgaaagggtcaaaattgagtttcatgatttgaggtattgggtttgtgtctctgatcaagatgggggccaatttgatgtgaaagaaatcctttgtaagattttagaggtggttactaaggagaaagttgataatagttccgcattggaattggtacaaagccaatttcaagagaagttaagaggaaagaagtacttccttgttcttgatgatgtatggaacgaggatcgtgagaagtggtttaaattggaagagttgttaatgttgggtcaagggggaagcaaggttgtagtgaccgcacgttcagagaagacagcaaatgtcatagggaaaagacatttttatacactggaatgtttgtcgccagattattcatggagcttatttgaaatgtcggcttttcagaaagggcatgagcaggaaaaccatgacgaactagttgatattgggaaaaagattgttgaaaaatgttataacaatccacttgctataacggtggtaggaagtcttctttatggagaggagataagtaagtggcggtcatttgaaatgagtgagttggccaaaattggcaatggggataataagattttgtcgatattgaagctcagttactacaatcttgcaaactctttgaagagttgttttagttattgtgcagtatttcccaaggatcataaaatagagaaggagatgttgattgacctttggatagcacaaggatatgttgtgccgttggatggtggtcaaagtatagaagatgctgccgaggaacattttgtaattttattacggagatgtttctttcaagatgtagtgaaggatgtatacggtgatgttgattctgttaaaatccacgacttgatgcacgatgtcgcccaagaagtggggagggaggaaatatgtgtagtgaatgctaatacaaagaacttgggtgataaaatccgtcatgtacatggtgatgtcaatagatatgcacaaagagtctctctgtgtagccataagattcgttcgtatattggtggtaattgtgaaaaacgttgggtggatacactaatagacaactggatgtgtcttaggatgttggacttgtcaaggccggatgttaaaaatttgcctaattcaataggtaaattgttgcacttgaggtatcttaacctgtcttgtaatgatgatctgttgatactccctgatgcaattacaagactgcataatttgcagacactgcttttaaaagattgcggaagtttaaaggagttgccaaaagatttttgcaaattggtcaaactgagacacttggatttaaggtattgttggcgtttgattggtatgccattgggaatggatatgctaactagtcttagagtactgccatactttgtggtgggtaggaagaaacaaagtgttgatgatgagctgaaagcccttaaaggcctcaccgagataaaaggctccattaatatcaaaatctgtgaaaattatagaatagttgaaggcatgaatgacacaggaggagctgggtatttgaagagcatgaaacatctcacgggggttgatattacatttgatggtggatgtgttaaccctgaagctgtgttggaaaccctagagccaccttcaaatatcaagaggttatctatagataattacgatggtacaacaattccagtatggggaagagcagagattaattgggcaatctccctctcacatcttgtcgacatttggttttgtggttgtagtaatttgcaggagatgccagtgctgagtaaactgcctcatttgaaatcactgaatctttttaagttttgtaagttagagtacatggagagtagaagcagcagcagtagcagtgacacagaagcagcaacaccagaattaccaacattcttcccttcccttgaaaaacttacactttggtatctggaaaagttgaagggtttggggaacaggagatcgagtagttttccccgcctctctgaattggaaatctgggaatgcccagatctaacgtggtttcctccttgtccaagccttaaaacgttgaaattggaaaaaaacaatgaagcgttgcaaataatagtaaaaataacaacaacaagaggtaaagaagaaaaagaagaagacaagaatgctggtgttggaaattcacaagatgatgacaatgtcaaattacggaaggcggaaatagacaatctgggttatctcaaatcactgcccacaaattgtctgactcacctcgacattacaataagagattccaaggagggggagggtgaatgggaagttggggaggcatttcagaagtgtgtatcttctttgagaaagctcagcataatcggaaatcacggaataaataaagtgaagagactgtctggaagaacagggttggagcatttcactctgttggactcactcaaattttcaaagatagaagaccaggaagatgagggcgaagacaacatcatattctggaaatcctttcctcaaaacctccgcagtttggaaattaaaggctcttgcaaaatgacaagtttgcccatggggatgcagtacttaacctccctccaaaccctccatctatcatattgtgatgaattgaattcccttccagaatggataagcagcttatcatctcttcaatccctgttcatatacaattgtccagccctgaaatcactaccagaagcaatgaagaacctcacctcccttcagagacttgagatacagcattgtccagacctagctgaaagatgcagaaaacccaacggggaggactatcccaaaattcaacacatccccaaaattgtaagtcattgcagaaagtaatttattcatttatatttattttatgcttagaatgatatacgcagtcgtcctttggtttccaatcttgaatttggtttttgttttctttctttgtttctttattcaacaccagtccatttatgattgattcattaaaaaaaggatggagttttatggatttgaagaagacaacgaattgagattcctggggttttttttttcgttggggttggttttcatgtatatgttgctgattaaataccagactgatgatgatgatgtgtttatgggttttaaatcagattaaatatatgggaaatgtaagttaattggggatgcacataaggtgtttgatgaaatgtctatgagaaatgttgtttcttggacttagaatgatatacactgtcgtcctttggtttccaatcttacatttggtttgtgttttcttagtttgtttctttaatcaacaccagcccattttttttaaactacctgcaactactaattttcatttaccctgtatctcaggaaatatggtagtaattctcatttactcaacactagcttgatcctgaacgcagccaaccttcaggttagaatccgccttactcatccttttgtcatgcattgttttaagttgttttgcttgcttgtgtaatcataattcatagtatacgattcatcattcactatgtctacaggcaagatattggaattgttcacgattccctgaagtttctttgtttttgttgataccaccatattgcagcttatagtgactaagttaatgaatgtttccaaaaaattagtcatataaattcttcttctctctctattacataaactctttttctctttctaacttatcatgttcatgcctaaaacttatacatgctcacatcattgttcgtttgagctgacttacttctgtaagagagctatctagttaacaactcttgtaactttttatttgctagtcagaacatggattggtgcaagcatgggaatttgctaacactctaccaaatcgattggagtttggacttagtttcaccagaagccatacccggacacttactggggactgtcaacaaagccgcattgtgatgtacttggatgtttcacgtgcctgaggtgcgagttacttggaagggaagcggtttatttaattgttttcctaagtagattttgcttacaagcttttacttttcacttgaaagggtttttcttgttttaagcttttcgaattagagttttcggttgcattaagagtagtcgtattagtcttttacctaaggaagactcttttttgtaattttcagactatgcaattcaagttttcgagtgttttcttgcttgtgtgattgtgagttggtgaattcgtctttcatacattttgagattatcagaagctttatgctccaccggtagtctagtaccttttctgttactgtacgtgcagggaagtaatctggtaccttctatatatatggaaaaacatacattatacattatgcaaaattcttacaggttagttacttcctggaacttcatttacactttgttttttttgttccattccctcggaagactattccctctgagaaatatgtaatgaacttctgtatgttgctgtttggttcctgttttaatcttcaattttcttgtatagttacagctgcatttacaatgaagtttaagcagacactctctttatatagtgcttctttctggagcaccgttgagctgtctgtggttgatcaccatctgctgccgagagattcagcaatcgcgtgtttgatcaggtaaaagtttttatgtcaatgtgtttttttttccgtttgatcaatttatgtctgtattcagattcttatcttcttacagtagcataacacattgtttctttcatttatgtaaactgtttcaagattacagagatgtatgcttcagtcgacattgatgataacttaagatagcattcctacaacagttgcaggcgcattctaactccggcaattctagttaggcaagaggagcattgccaatacctgccacctctgggatttactataccagggttgaagtttatggaagacaccagctatgcacaagccttcaaggggtcatcctacataacaagttgaaccaaccaattgcttgttggttcagtggtaattggagctgaatttggtagggatggcccatgttcgatccccacaacaacaattgggaggggactggaacctatccacacgaactccgccctgaatccggattagtcctaagggtgaacggggtgctaacaccaaaaaaaaaaaacataacaagttgaaccaaacatactttgtttgaattgaagatttagtgatttcatttgatcgattgagatgtcttattataagcgtatatgctcttggatttggccacttaggtgttgtttgacaattggtcattaactcgcttttatattttcttttctcttaggaaaggtgatcctgataatttatattggaacactttttttttctctcactagcttt

SEQ ID NO: 12 - DNA, 3450 bp, Spinacia oleracea
  misc_feature 1..3450: note = Coding sequence of the alpha-WOLF 27 allele
  source 1..3450: mol_type = genomic DNA
atggccgaaatcggatactcggtttgtgcgaaactcatcgaagtgattggcagtgagctgatcaaagagatttgcgacacatggggttacaaatctcttcttgaggacctcaacaaaactgtattgacggtcaggaacgttctcattcaggccggggtgatgcgggagcttactagtgaacaacaaggtttcattgcagaccttaaagatgttgtttatgatgctgatgacttgttcgacaagttactcactcgtgctgagcgaaaacagattgatggaaacgaaatctctgaaaaggtacgtcgtttcttttcctctagtaacaagatcggtcaagcttactacatgtctcgtaaggttaaggaaattaagaagcagttggatgaaattgttgataggcatacaaaatttgggtttagtgctgagtttatacctgtttgtagggaaagggggaacgagagggaaacacgttcatatatagatgtcaagaatattcttgggagggataaagataagaatgatatcatagataggttgcttaatcgtaatgataatgaagcttgtagtttcctgaccatagtgggagcgggaggattgggaaaaactgctcttgcccaacttgtgttcaatgatgaaagggtcaaaattgagtttcatgatttgaggtattgggtttgtgtctctgatcaagatgggggccaatttgatgtgaaagaaatcctttgtaagattttagaggtggttactaaggagaaagttgataatagttccgcattggaattggtacaaagccaatttcaagagaagttaagaggaaagaagtacttccttgttcttgatgatgtatggaacgaggatcgtgagaagtggtttaaattggaagagttgttaatgttgggtcaagggggaagcaaggttgtagtgaccgcacgttcagagaagacagcaaatgtcatagggaaaagacatttttatacactggaatgtttgtcgccagattattcatggagcttatttgaaatgtcggcttttcagaaagggcatgagcaggaaaaccatgacgaactagttgatattgggaaaaagattgttgaaaaatgttataacaatccacttgctataacggtggtaggaagtcttctttatggagaggagataagtaagtggcggtcatttgaaatgagtgagttggccaaaattggcaatggggataataagattttgtcgatattgaagctcagttactacaatcttgcaaactctttgaagagttgttttagttattgtgcagtatttcccaaggatcataaaatagagaaggagatgttgattgacctttggatagcacaaggatatgttgtgccgttggatggtggtcaaagtatagaagatgctgccgaggaacattttgtaattttattacggagatgtttctttcaagatgtagtgaaggatgtatacggtgatgttgattctgttaaaatccacgacttgatgcacgatgtcgcccaagaagtggggagggaggaaatatgtgtagtgaatgctaatacaaagaacttgggtgataaaatccgtcatgtacatggtgatgtcaatagatatgcacaaagagtctctctgtgtagccataagattcgttcgtatattggtggtaattgtgaaaaacgttgggtggatacactaatagacaactggatgtgtcttaggatgttggacttgtcaaggccggatgttaaaaatttgcctaattcaataggtaaattgttgcacttgaggtatcttaacctgtcttgtaatgatgatctgttgatactccctgatgcaattacaagactgcataatttgcagacactgcttttaaaagattgcggaagtttaaaggagttgccaaaagatttttgcaaattggtcaaactgagacacttggatttaaggtattgttggcgtttgattggtatgccattgggaatggatatgctaactagtcttagagtactgccatactttgtggtgggtaggaagaaacaaagtgttgatgatgagctgaaagcccttaaaggcctcaccgagataaaaggctccattaatatcaaaatctgtgaaaattatagaatagttgaaggcatgaatgacacaggaggagctgggtatttgaagagcatgaaacatctcacgggggttgatattacatttgatggtggatgtgttaaccctgaagctgtgttggaaaccctagagccaccttcaaatatcaagaggttatctatagataattacgatggtacaacaattccagtatggggaagagcagagattaattgggcaatctccctctcacatcttgtcgacatttggttttgtggttgtagtaatttgcaggagatgccagtgctgagtaaactgcctcatttgaaatcactgaatctttttaagttttgtaagttagagtacatggagagtagaagcagcagcagtagcagtgacacagaagcagcaacaccagaattaccaacattcttcccttcccttgaaaaacttacactttggtatctggaaaagttgaagggtttggggaacaggagatcgagtagttttccccgcctctctgaattggaaatctgggaatgcccagatctaacgtggtttcctccttgtccaagccttaaaacgttgaaattggaaaaaaacaatgaagcgttgcaaataatagtaaaaataacaacaacaagaggtaaagaagaaaaagaagaagacaagaatgctggtgttggaaattcacaagatgatgacaatgtcaaattacggaaggcggaaatagacaatctgggttatctcaaatcactgcccacaaattgtctgactcacctcgacattacaataagagattccaaggagggggagggtgaatgggaagttggggaggcatttcagaagtgtgtatcttctttgagaaagctcagcataatcggaaatcacggaataaataaagtgaagagactgtctggaagaacagggttggagcatttcactctgttggactcactcaaattttcaaagatagaagaccaggaagatgagggcgaagacaacatcatattctggaaatcctttcctcaaaacctccgcagtttggaaattaaaggctcttgcaaaatgacaagtttgcccatggggatgcagtacttaacctccctccaaaccctccatctatcatattgtgatgaattgaattcccttccagaatggataagcagcttatcatctcttcaatccctgttcatatacaattgtccagccctgaaatcactaccagaagcaatgaagaacctcacctcccttcagagacttgagatacagcattgtccagacctagctgaaagatgcagaaaacccaacggggaggactatcccaaaattcaacacatccccaaaattgaaatatggtag

SEQ ID NO: 13 - protein, 1149 aa, Spinacia oleracea
  REGION 1..1149: note = Amino acid sequence of the alpha-WOLF 27 allele
MAEIGYSVCAKLIEVIGSELIKEICDTWGYKSLLEDLNKTVLTVRNVLIQAGVMRELTSEQQGFIADLKDVVYDADDLFDKLLTRAERKQIDGNEISEKVRRFFSSSNKIGQAYYMSRKVKEIKKQLDEIVDRHTKFGFSAEFIPVCRERGNERETRSYIDVKNILGRDKDKNDIIDRLLNRNDNEACSFLTIVGAGGLGKTALAQLVFNDERVKIEFHDLRYWVCVSDQDGGQFDVKEILCKILEVVTKEKVDNSSALELVQSQFQEKLRGKKYFLVLDDVWNEDREKWFKLEELLMLGQGGSKVVVTARSEKTANVIGKRHFYTLECLSPDYSWSLFEMSAFQKGHEQENHDELVDIGKKIVEKCYNNPLAITVVGSLLYGEEISKWRSFEMSELAKIGNGDNKILSILKLSYYNLANSLKSCFSYCAVFPKDHKIEKEMLIDLWIAQGYVVPLDGGQSIEDAAEEHFVILLRRCFFQDVVKDVYGDVDSVKIHDLMHDVAQEVGREEICVVNANTKNLGDKIRHVHGDVNRYAQRVSLCSHKIRSYIGGNCEKRWVDTLIDNWMCLRMLDLSRPDVKNLPNSIGKLLHLRYLNLSCNDDLLILPDAITRLHNLQTLLLKDCGSLKELPKDFCKLVKLRHLDLRYCWRLIGMPLGMDMLTSLRVLPYFVVGRKKQSVDDELKALKGLTEIKGSINIKICENYRIVEGMNDTGGAGYLKSMKHLTGVDITFDGGCVNPEAVLETLEPPSNIKRLSIDNYDGTTIPVWGRAEINWAISLSHLVDIWFCGCSNLQEMPVLSKLPHLKSLNLFKFCKLEYMESRSSSSSSDTEAATPELPTFFPSLEKLTLWYLEKLKGLGNRRSSSFPRLSELEIWECPDLTWFPPCPSLKTLKLEKNNEALQIIVKITTTRGKEEKEEDKNAGVGNSQDDDNVKLRKAEIDNLGYLKSLPTNCLTHLDITIRDSKEGEGEWEVGEAFQKCVSSLRKLSIIGNHGINKVKRLSGRTGLEHFTLLDSLKFSKIEDQEDEGEDNIIFWKSFPQNLRSLEIKGSCKMTSLPMGMQYLTSLQTLHLSYCDELNSLPEWISSLSSLQSLFIYNCPALKSLPEAMKNLTSLQRLEIQHCPDLAERCRKPNGEDYPKIQHIPKIEIW

SEQ ID NO: 14 - protein, 9 aa, Spinacia oleracea
  REGION 1..9: note = motif
DQEDEGEDN

SEQ ID NO: 15 - DNA, 30 bp, Spinacia oleracea
  misc_feature 1..30: note = standard amplification sequence forward primer
  source 1..30: mol_type = genomic DNA
gcagtcgaacatgtagctgactcaggtcac

SEQ ID NO: 16 - DNA, 30 bp, Spinacia oleracea
  misc_feature 1..30: note = standard amplification sequence reverse primer
  source 1..30: mol_type = genomic DNA
tggatcacttgtgcaagcatcacatcgtag

(19) United States
(12) Patent Application Publication
(10) Pub. No.: US 2023/0329169 A1
(43) Pub. Date: Oct. 19, 2023

(54) PERONOSPORA RESISTANCE IN SPINACIA OLERACEA

(71) Applicant: RIJK ZWAAN ZAADTEELT EN ZAADHANDEL B.V., De Lier (NL)

(72) Inventors: Erik DEN BOER, De Lier (NL); Raoul Jacobus Johannes Maria FRIJTERS, De Lier (NL)

(21) Appl. No.: 18/306,580

(22) Filed: Apr. 25, 2023

Related U.S. Application Data
(63) Continuation-in-part of application No. PCT/EP2021/080275, filed on Nov. 1, 2021.
Application No. PCT/EP2021/080275 is a continuation-in-part of application No. PCT/EP2020/080597, filed on Oct. 30, 2020.
Application No. PCT/EP2020/080597 is a continuation-in-part of application No. 17/085,860, filed on Oct. 30, 2020, now Pat. No. 11,473,102.

(51) Int. Cl. A01H 1/00; C12Q 1/6895; A01H 5/12; A01H 6/02

(52) U.S. Cl. CPC A01H 1/1255; C12Q 1/6895; A01H 5/12; A01H 6/028; C12Q 2600/13; C12Q 2600/158

(57) ABSTRACT

The present invention relates to an allele designated alpha-WOLF 27 which confers resistance to at least one Peronospora effusa race, wherein the protein encoded by said allele is a CC-NBS-LRR protein that comprises in its amino acid sequence: a) the motif “MAEIGYSVC” (SEQ ID NO: 1) at its N-terminus; and b) the motif “KWMCLR” (SEQ ID NO: 2); and wherein the LRR domain of the protein has in order of increased preference at least 95%, 95.5%, 96%, 96.5%, 97%, 97.5%, 98%, 98.5%, 99%, 99.5%, 100% sequence similarity to SEQ ID NO: 10. The allele when present in a spinach plant homozygously confers complete resistance to at least Peronospora effusa race Pe: 7, Pe: 8, Pe: 9, Pe: 11, Pe: 12, Pe: 13, Pe: 14, Pe: 15, Pe: 17.

### RELATED APPLICATIONS AND INCORPORATION BY REFERENCE

This application is a continuation-in-part application of international patent application Serial No. PCT/EP2021/080275 filed 1 Nov. 2021, which published as PCT Publication No. WO 2022/090543 on 5 May 2022, which claims benefit of international patent application Serial No. PCT/EP2020/080597 filed 30 Oct. 2020 and U.S. patent application Ser. No. 17/085,860 filed 30 Oct. 2020.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE STATEMENT

The instant application contains a Sequence Listing which has been submitted electronically and is hereby incorporated by reference in its entirety. Said XML copy, was created Apr. 20, 2023, is named Y7954-00575SL.xml and is 37,911 bytes in size.

### FIELD OF THE INVENTION

The invention relates to a gene capable of conferring resistance to a spinach plant against one or more Peronospora effusa races. The invention also relates to a spinach plant, to propagation material of said spinach plant, to a cell of said spinach plant, and to seed of said spinach plant carrying the gene. The invention further relates to a method of producing a spinach plant carrying the gene and to the use of the gene in breeding to confer resistance against Peronospora effusa.

### BACKGROUND OF THE INVENTION

Downy mildew (Peronospora effusa) is a major threat for spinach growers because it directly affects the harvested leaves. In spinach (Spinacia oleracea), downy mildew is caused by the oomycete Peronospora effusa (formerly known as Peronospora farinosa f sp. spinaciae). Infection makes the leaves unsuitable for sale and consumption, as it manifests itself phenotypically as yellow lesions on the older leaves, and on the abaxial leaf surface a greyish fungal growth can be observed. The infection can spread very rapidly, and it can occur both in glasshouse cultivation and in soil cultivation. The optimal temperature for formation and germination of Peronospora effusa spores is 9 to 12° C., and it is facilitated by a high relative humidity. When spores are deposited on a humid leaf surface they can readily germinate and infect the leaf. Fungal growth is optimal between 8 and 20° C. and a relative humidity of ≥80%, and within 6 and 13 days after infection mycelium growth can be observed. Oospores of P. effusa can survive in the soil for up to 3 years, or as mycelium in seeds or living plants.

To date 19 pathogenic races of spinach downy mildew (Pe) have been officially identified and characterized, and many new candidates are observed in the field. The 17 officially recognized races of Peronospora effusa, are designated Pe:1 to Pe:19 (Pe:1 to Pe:17 were formerly known as Pfs:1 to Pfs:17; Irish et al. Phtypathol. Vol. 98 pg. 894-900, 2008; Plantum NL (Dutch association for breeding, tissue culture, production and trade of seed and young plants) press release, “Benoeming van Pfs: 14, een nieuwe fysio van valse meeldauw in spinazie”, Sep. 19, 2012; Report Jim Correl (Univ. Arkansas) and Steven Koike (UC Cooperative Extension, Monterey County), “Race Pfs: 14—Another new race of the spinach downy mildew pathogen”, Sep. 18, 2012; Plantum NL press release, “Denomination of Pfs: 15, a new race of downy mildew in spinach”, Sep. 2, 2014; Plantum NL press release, “Denomination of Pfs: 16, a new race of downy mildew in spinach, Mar. 15, 2016; Plantum NL press release, Denomination of Pfs: 17, a new race of downy mildew in spinach”, Apr. 16, 2018; Plantum NL press release, “Denomination of Pe: 18 and 19, two new races of downy mildew in spinach”, Apr. 15, 2021).

All 19 officially recognized Pe races are publicly available from the Department of Plant Pathology, University of Arkansas, Fayetteville, AR 72701, USA, and also from NAK Tuinbouw, Sotaweg 22, 2371 GD Roelofarendsveen, the Netherlands.

Especially the latest identified Peronospora races can break the resistance of many spinach varieties that are currently used commercially worldwide, and they thus pose a serious threat to the productivity of the spinach industry. Therefore, it is crucial to stay at the forefront of developments in this field, as Peronospora continuously develops the ability to break the resistances that are present in commercial spinach varieties. For this reason new resistance genes against downy mildew are very valuable assets, and they form an important research focus in breeding and particular in spinach and lettuce breeding. One of the main goals of spinach breeders is to rapidly develop spinach varieties with a resistance to as many Peronospora races as possible, including the latest identified races, before these races become wide-spread and pose a threat to the industry.

In commercial spinach varieties resistance against downy mildew is usually caused by so-called R-genes. R-gene mediated resistance is based on the ability of a plant to recognize the invading pathogen. In many cases this recognition occurs after the pathogen has established the first phases of interaction and transferred a so called pathogenicity (or avirulence) factor into the plant cell. These pathogenicity factors interact with host components in order to establish conditions which are favorable for the pathogen to invade the host and thereby cause disease. When a plant is able to recognize the events triggered by the pathogenicity factors a resistance response can be initiated. In many different plant pathogen interaction systems such as the interaction of spinach with different downy mildew strains, the plant initiates these events only after specific recognition of the invading pathogen.

Co-evolution of plant and pathogen has led to an arms race in which a R-gene mediated resistance is sometimes overcome as a consequence of the capability of the pathogen to interact with and modify alternative host targets or the same targets in a different way, such that the recognition is lost and infection can be established successfully resulting in disease. In order to re-establish resistance in a plant, a new R-gene has to be introduced which is able to recognize the mode of action of an alternative pathogenicity factor.

Despite the fact that the durability of R-genes is relatively low, R-genes are in spinach still the predominant form of defense against downy mildew. This is mainly due to the fact that it is the only form of defense that gives absolute resistance. So far plant breeders have been very successful in generating downy mildew resistant spinach varieties by making use of resistance genes residing in the wild germplasm of the crop species. Even though R-genes are extensively used in spinach breeding, until now not much is known of these R-genes.

Citation or identification of any document in this application is not an admission that such document is available as prior art to the present invention.

### SUMMARY OF THE INVENTION

Only recently it was discovered that the R-genes officially recognized in spinach are in fact all different alleles of the two tightly linked genes, the alpha- and the beta-WOLF genes. This was also the first time that R-genes, or better R-alleles were for the first time characterized at the molecular level, i.e. their nucleotide and amino acid sequence was determined. Although this provides the breeder with tools that increase the efficiency of detecting and selecting R-alleles, adequately responding to newly emerging downy mildew races is still crucial for developing commercially successful spinach varieties. Therefore, it is the object of the invention to provide a new resistance allele conferring resistance to a newly emerged downy mildew isolate and to provide molecular biological tools for identifying this new resistance allele.

In the research leading to the present invention, a new allelic variant of the Alpha-WOLF gene as described in WO2018059651 was found. The alpha-WOLF gene encodes a protein that belongs to the CC-NBS-LRR family (Coiled Coil—Nucleotide Binding Site—Leucine-Rich Repeat). Depending on the allelic variant (or the allelic variants) that is (are) present in a spinach plant, said plant will produce a variant of the WOLF protein that confers a certain resistance profile to pathogenic races of Peronospora effusa.

In the context of this invention the term “allele” or “allelic variant” is used to designate a version of the gene that is linked to a specific phenotype, i.e. resistance profile. It was found that a spinach plant may carry one or two WOLF genes. Each of these two WOLF genes encompasses multiple alleles, each allele conferring a particular resistance profile. In the context of this invention an allele or allelic variant is a nucleic acid.

The beta WOLF gene is located on scaffold12735 (sequence: GenBank: KQ143339.1), at position 213573-221884. In case the spinach plant also carries or only carries the alpha-WOLF gene, the alpha-WOLF gene is located at approximately the same location as where the beta-WOLF gene is located on scaffold12735 in the Viroflay genome assembly.

The newly found alpha-WOLF allele provides resistance to at least downy mildew race Pe:14, Pe:15 and Pe:17.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

Deposit

Seeds of a plant that comprises the alpha-WOLF 27 allele of the invention in its genome were deposited with NCIMB Ltd, Ferguson Building, Craibstone Estate, Bucksburn, Aberdeen AB21 9YA, UK, on 9 Oct. 2020, under accession number NCIMB 43668. The deposit was made and accepted pursuant to the terms of the Budapest Treaty. Upon issuance of a patent, all restrictions upon the deposit will be removed, and the deposit is intended to meet the requirements of 37 CFR §§ 1.801-1.809. The deposit will be irrevocably and without restriction or condition released to the public upon the issuance of a paten and for the enforceable life of the patent. The deposit will be maintained in the depository for a period of 30 years, or 5 years after the last request, or for the effective life of the patent, whichever is longer, and will be replaced if necessary during that period.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1. Resistance profile conferred by the alpha-WOLF 27 allele when homozygously present in a spinach plant. A “−” means complete resistance against a particular downy mildew race; “(−)” means intermediate resistance against a particular downy mildew race; “+” means that the allele confers no resistance and would cause a plant only carrying the alpha-WOLF 27 allele to be fully susceptible for that particular downy mildew race; “nt” means that it has not been tested against that isolate.

FIG. 2. Sequence Information.

FIG. 3. Differential set of spinach downy mildew races and the resistance of various spinach varieties (hybrids) to each one of these pathogenic races. A susceptible reaction is scored as “+” (indicating a successful infection by the fungus, with sporulation occurring on the entire cotyledon), and resistance is depicted as “−” (absence of sporulation on the cotyledons). An intermediate resistance response is indicated as “(−)”, which in practice means a slightly level of infection (with only symptoms of chlorosis, or sporulation only occurring on the tips of the cotyledons in the differential seedling test).

### DETAILED DESCRIPTION OF THE INVENTION

A genome assembly for spinach variety Viroflay—which is susceptible to all known pathogenic races of Peronospora effusa—is publicly available (Spinacia oleracea cultivar SynViroflay, whole genome shotgun sequencing project; Bioproject: PRJNA41497; GenBank: AYZV00000000.2; BioSample: SAMN02182572, see also Dohm et al, 2014, Nature 505: 546-549). In this genome assembly for Viroflay, the beta-WOLF gene is located on scaffold12735 (sequence: GenBank: KQ143339.1), at position 213573-221884. The sequence covered by this interval comprises the entire genomic sequence of the beta-WOLF gene of Viroflay, plus 2000 basepairs sequence upstream from the gene, plus the sequence downstream from the gene, up to the locus of the neighbouring gene that is situated downstream from the WOLF gene. Spinach variety Viroflay only possesses a single WOLF gene, namely a beta-WOLF gene, but most other spinach lines harbor a single alpha-type WOLF gene at the same location in the genome. Other spinach lines harbor two WOLF genes at approximately the same location in the genome. In such cases, the two WOLF genes are positioned adjacent to each other. In most spinach lines that harbor two WOLF genes, one of said WOLF genes belongs to the alpha-type, and the other WOLF gene belongs to the beta-type. It was observed that this allelic variation in the WOLF locus is responsible for differences in resistance to pathogenic races of Peronospora effusa.

The difference between an allele of an alpha-WOLF gene and an allele of a beta-WOLF gene lies in the presence of specific conserved amino acid motifs in the encoded protein sequence. As mentioned above, all WOLF proteins possess—from N- to C-terminus—the following domains that are generally known in the art: a coiled coil domain (RX-CC-like, cd14798), an NBS domain (also referred to as “NB-ARC domain”, pfam00931; van der Biezen & Jones, 1998, Curr. Biol. 8: R226-R228), and leucine-rich repeats (IPR032675) which encompass the LRR domain. In addition, all WOLF proteins comprise in their amino acid sequence the motif “MAEIGYSVC” (SEQ ID NO: 1) at the N-terminus. In addition to this, all alpha-WOLF proteins comprise the motif “KWMCLR” (SEQ ID NO: 2) in their amino acid sequence, whereas all beta-WOLF proteins comprise the motif “HVGCVVDR” (SEQ ID NO: 3) in their amino acid sequence.

The present invention relates to a new Peronospora effusa resistance conferring allele of the alpha-WOLF gene designated alpha-WOLF 27.

In particular, the invention relates to a Peronospora effusa resistance conferring allele designated alpha-WOLF 27 wherein the protein encoded by said allele is a CC-NBS-LRR protein that comprises in its amino acid sequence: a) the motif “MAEIGYSVC” (SEQ ID NO: 1) at its N-terminus; and b) the motif “KWMCLR”(SEQ ID NO: 2); and wherein the LRR domain of the protein has in order of increased preference at least 95%, 95.3%, 95.5%, 95.8%, 96%, 96.3%, 96.5%, 96.8%, 97%, 97.3%, 97.5%, 97.8%, 98%, 98.3%, 98.5%, 98.8%, 99%, 99.3%, 99.5%, 99.8%, 100% sequence identity to SEQ ID NO: 10. Optionally, the alpha-WOLF 27 allele further comprises an additional motif in its amino acid sequence, namely “DQEDEGEDN” (SEQ ID NO: 18).

The invention further relates to a Peronospora effusa resistance conferring allele designated alpha-WOLF 27 wherein the protein encoded by said allele is a CC-NBS-LRR protein that comprises in its amino acid sequence: a) the motif “MAEIGYSVC” (SEQ ID NO: 1) at its N-terminus; and b) the motif “KWMCLR” (EQ ID NO: 2); and wherein the LRR domain of the protein has in order of increased preference at least 96%, 96.3%, 96.5%, 96.8%, 97%, 97.3%, 97.5%, 97.8%, 98%, 98.3%, 98.5%, 98.8%, 99%, 99.3%, 99.5%, 99.8%, 100% sequence similarity to SEQ ID NO: 10. Optionally, the alpha-WOLF 27 allele further comprises an additional motif in its amino acid sequence, namely “DQEDEGEDN” (SEQ ID NO: 18).

The invention also relates to an alpha-WOLF 27 allele having an LRR domain which has a sequence that in order in order of increased preference has at least 95%, 95.3%, 95.5%, 95.8%, 96%, 96.3%, 96.5%, 96.8%, 97%, 97.3%, 97.5%, 97.8%, 98%, 98.3%, 98.5%, 98.8%, 99%, 99.1%, 99.2%, 99.3%, 99.4%, 99.5%, 99.6%, 99.7%, 99.8%, 99.9%, 100% sequence identity to SEQ ID NO: 9.

For the purpose of this invention, the LRR domain of the protein of the alpha-WOLF 27 allele is defined as the amino acid sequence that in order of increased preference has at least 95%, 95.3%, 95.5%, 95.8%, 96%, 96.3%, 96.5%, 96.8%, 97%, 97.3%, 97.5%, 97.8%, 98%, 98.3%, 98.5%, 98.8%, 99%, 99.1%, 99.2%, 99.3%, 99.4%, 99.5%, 99.6%, 99.7%, 99.8%, 99.9%, 100% sequence identity to SEQ ID NO: 10.

For the purpose of this invention, the LRR domain of the protein of the alpha-WOLF 27 allele is defined as the amino acid sequence that in order of increased preference has at least 96%, 96.3%, 96.5%, 96.8%, 97%, 97.3%, 97.5%, 97.8%, 98%, 98.3%, 98.5%, 98.8%, 99%, 99.1%, 99.2%, 99.3%, 99.4%, 99.5%, 99.6%, 99.7%, 99.8%, 99.9%, 100% sequence similarity to SEQ ID NO: 10.

The skilled person is familiar with methods for the calculation of sequence similarity and sequence identity. Sequence similarity for an amino acid sequence is calculated using EMBOSS stretcher 6.6.0 (www.ebi.ac.uk/Tools/psa/emboss_stretcher), using the EBLOSUM62 matrix with settings Gap open penalty: 12 and Gap extend penalty: 2. In case of DNA, sequence similarity is calculated using the DNA full matrix with settings Gap open penalty:16 and Gap extend penalty: 4.

The LRR domain of the alpha-WOLF 27 allele as defined herein can be determined by amplifying and sequencing the genomic DNA encoding for the amino acid sequence of LRR domain using specific primers, and subsequently translating the DNA sequence into an amino acid sequence, thereby applying common sense in choosing the correct reading frame. The skilled person is capable of doing this, using freely available online bioinformatics tools such as can be found here: http://web.expasy.org/translate/.

The genomic sequence of a LRR domain of an alpha-WOLF gene such as alpha-WOLF 27 can be amplified using a primer pair having a forward primer which is a nucleic acid molecule having the sequence of SEQ ID NO: 4 and a reverse primer which is a nucleic acid molecule having the sequence of SEQ ID NO: 5.

The invention also relates to a nucleic acid molecule which confers resistance to at least one Peronospora effusa race, wherein the protein encoded by said nucleic acid molecule is a CC-NBS-LRR protein that comprises in its amino acid sequence: a) the motif “MAEIGYSVC” (SEQ ID NO: 1) at its N-terminus; and b) the motif “KWMCLR” (SEQ ID NO: 2); and wherein the LRR domain of the protein has in order of increased preference at least 95%, 95.3%, 95.5%, 95.8%, 96%, 96.3%, 96.5%, 96.8%, 97%, 97.3%, 97.5%, 97.8%, 98%, 98.3%, 98.5%, 98.8%, 99%, 99.3%, 99.5%, 99.8%, 100% sequence identity to SEQ ID NO: 10. Optionally this nucleic acid molecule is an isolated nucleic acid molecule.

The invention also relates to a nucleic acid molecule which confers resistance to at least one Peronospora effusa race, wherein the protein encoded by said nucleic acid molecule is a CC-NBS-LRR protein that comprises in its amino acid sequence: a) the motif “MAEIGYSVC” (SEQ ID NO: 1) at its N-terminus; and b) the motif “KWMCLR” (SEQ ID NO: 2); and wherein the LRR domain of the protein has in order of increased preference at least 96%, 96.3%, 96.5%, 96.8%, 97%, 97.3%, 97.5%, 97.8%, 98%, 98.3%, 98.5%, 98.8%, 99%, 99.3%, 99.5%, 99.8%, 100% sequence similarity to SEQ ID NO: 10. Optionally this nucleic acid molecule is an isolated nucleic acid molecule.

PCR conditions for amplifying the LRR domain-encoding region of an alpha-WOLF gene using primers having SEQ ID NO: 4 and SEQ ID NO: 5 are, using Platinum Taq enzyme (Thermo Fisher Scientific): 3 minutes at 95° C. (initial denaturing step); 40 amplification cycles, each cycle consisting of: 30 seconds denaturation at 95° C., 30 seconds annealing at 60° C., and 30 seconds extension at 72° C.; 2 minutes at 72° C. (final extension step).

The LRR domain of a beta-WOLF gene, e.g. the null allele as present in variety Viroflay, can be amplified using a forward primer which is a nucleic acid molecule having the sequence of SEQ ID NO: 6 and a reverse primer which is a nucleic acid molecule having the sequence of SEQ ID NO: 5.

PCR conditions for amplifying the LRR domain-encoding region of a beta-WOLF gene using primers having SEQ ID NO: 5 and SEQ ID NO: 6 are as follows, using Platinum Taq enzyme (Thermo Fisher Scientific): 3 minutes at 95° C. (initial denaturing step); 40 amplification cycles, each cycle consisting of: 30 seconds denaturation at 95° C., 50 seconds annealing at 58° C. and 50 seconds extension at 72° C.; 2 minutes at 72° C. (final extension step).

Therefore, the invention also relates to a primer pair for amplifying the LRR domain of an alpha-WOLF gene, more in particular for amplifying the LRR domain of an alpha-WOLF 27 allele wherein the forward primer is a nucleic acid molecule having the sequence of SEQ ID NO: 4 and the reverse primer which is a nucleic acid molecule having the sequence of SEQ ID NO: 5. The primers disclosed herein have been specifically designed for selectively amplifying part of a WOLF gene, and not of any other CC-NBS-LRR protein-encoding genes.

The invention relates to an alpha-WOLF 27 allele which has a coding sequence that in order of increased preference has at least 95%, 95.5%, 96%, 96.5%, 97%, 97.5%, 97.8%, 98%, 98.3%, 98.5%, 98.8%, 99%, 99.1%, 99.2%, 99.3%, 99.4%, 99.5%, 99.6%, 99.7%, 99.8%, 99.9%, 100% sequence identity to SEQ ID NO: 12.

In a further aspect of the invention the alpha-WOLF 27 allele encodes for a protein having an amino acid sequence which in order of increased preference has at least 95%, 95.3%, 95.5%, 95.8%, 96%, 96.3%, 96.5%, 96.8%, 97%, 97.3%, 97.5%, 97.8%, 98%, 98.3%, 98.5%, 98.8%, 99%, 99.1%, 99.2%, 99.3%, 99.4%, 99.5%, 99.6%, 99.7%, 99.8%, 99.9%, 100% sequence identity to SEQ ID NO: 13.

In a further aspect of the invention the alpha-WOLF 27 allele encodes for a protein having an amino acid sequence which in order of increased preference has at least 95%, 95.3%, 95.5%, 95.8%, 96%, 96.3%, 96.5%, 96.8%, 97%, 97.3%, 97.5%, 97.8%, 98%, 98.3%, 98.5%, 98.8%, 99%, 99.1%, 99.2%, 99.3%, 99.4%, 99.5%, 99.6%, 99.7%, 99.8%, 99.9%, 100% sequence similarity to SEQ ID NO: 13.

The alpha-WOLF 27 allele when present in a spinach plant confers complete resistance to at least one of the 19 officially recognized Peronospora effusa races. In a further embodiment, the alpha-WOLF 27 allele when present in a spinach plant confers complete resistance to at least two of the 19 officially recognized Peronospora effusa races. In a further embodiment, the alpha-WOLF 27 allele when present in a spinach plant confers complete resistance in order of increased preference to at least two, three, four, five, six, seven, eight, nine, ten, eleven, twelve, thirteen, fourteen, fifteen, sixteen or seventeen of the nineteen officially recognized Peronospora effusa races.

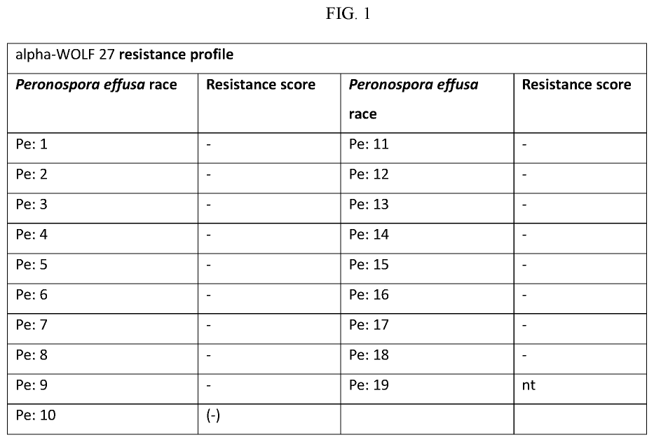

The alpha-WOLF 27 allele when homozygously present in a spinach plant confers complete resistance to at least the officially recognized Peronospora effusa races Pe: 7, Pe: 8, Pe: 9, Pe: 11, Pe: 12, Pe: 13, Pe: 14, Pe: 15, Pe: 17. More in particular, the alpha-WOLF 27 allele when homozygously present in a spinach plant confers complete resistance to at least the officially recognized Peronospora effusa races Pe: 1, Pe: 2, Pe: 3, Pe: 4, Pe: 5, Pe: 6, Pe: 7, Pe: 8, Pe: 9, Pe: 11, Pe: 12, Pe: 13, Pe: 14, Pe: 15, Pe: 17 (see FIG. 1). The alphaWOLF 27 allele also when homozygously present in a spinach plant confers complete resistance to at least the officially recognized Peronospora effusa races Pe:16 and Pe:18. Furthermore, the alphaWOLF 27 allele when homozygously present in a spinach plant confers intermediate resistance to Peronospora effusa race Pfs:10.

The resistance of a spinach plant against one or more races of Peronospora effusa can be determined using a seedling test. Herein, a seedling test is defined as a test wherein spinach plants are planted in trays containing growth medium, fertilized twice a week after seedling emergence. Plants are inoculated at the first true leaf stage with a sporangial suspension having a concentration of approximately 2.5×10⁵/ml of one of the pathogenic races of Peronospora effusa or isolates to be tested. Thirty plants per race are tested. The inoculated plants are placed in a dew chamber at 18° C. with 100% relative humidity for a 24 h period, and then moved to a growth chamber at 18° C. with a 12 h photoperiod for 6 days. After 6 days, the plants are returned to the dew chamber for 24 h to induce sporulation, and subsequently scored for a disease reaction.

As used herein, a plant is completely resistant against a Peronospora effusa race when a plant shows no symptoms in the seedling test described herein.

As used herein, a plant is intermediately resistant against a Peronospora effusa race when a plant shows only symptoms of chlorosis, or sporulation occurring only on the tips of the cotyledons in the seedling test described herein.

As used herein, a plant is susceptible to an isolate of a Peronospora effusa race when a plant shows more than only symptoms of chlorosis, or when sporulation occurs on area larger than only the tips of the cotyledons in the seedling test described herein.

Another aspect of the invention relates to a spinach plant, comprising the alpha-WOLF 27 allele of invention, of which a representative sample of seed was deposited with the NCIMB under accession number NCIMB 43668.

In a further embodiment the plant of the invention which comprises the alpha-WOLF 27 allele is an agronomically elite spinach plant. In the context of this invention an agronomically elite spinach plant is a plant having a genotype that results into an accumulation of distinguishable and desirable agronomic traits which allow a producer to harvest a product of commercial significance, preferably the agronomically elite spinach plant comprising the alpha-WOLF 27 allele is a plant of an inbred line or a hybrid.

As used herein, a plant of an inbred line is a plant of a population of plants that is the result of three or more rounds of selfing, or backcrossing; or which plant is a double haploid. An inbred line may e.g. be a parent line used for the production of a commercial hybrid.

As used herein, a hybrid plant is a plant which is the result of a cross between two different plants having different genotypes. More in particular, a hybrid plant is the result of a cross between plants of two different inbred lines, such a hybrid plant may e.g. be a plant of an F₁ hybrid variety.

A plant carrying the alpha-WOLF 27 allele in heterozygous form may further comprise a beta-WOLF 0 allele as e.g. present in variety Viroflay wherein the beta-WOLF 0 allele does not confer any resistance to downy mildew. However, a plant heterozygous for the alpha-WOLF 27 allele may further comprise an allele of the alpha/beta-WOLF gene that does provide resistance to downy mildew. Preferably, such an allele would complement the alpha-WOLF 27 allele such that the spinach plant will be at least intermediately resistant to one or more other races to which the alpha-WOLF 27 allele does not provide resistance. Most preferably the other allele of the alpha/beta-WOLF gene complements the alpha-WOLF 27 allele such that the plant is resistant to Peronospora effusa races Pe:1 to Pe:19. In one embodiment such a plant is an agronomically elite plant.

Alternatively, the resistance profile of a plant carrying the alpha-WOLF 27 allele is complemented by a resistance conferring allele of a totally different gene. Examples of such genes are e.g. DMR1 as described in U.S. Pat. No. 8,354,570, DMR6 as described in U.S. Pat. No. 9,121,029 and p10 as described in U.S. Pat. No. 10,226,016.

The invention thus relates to a spinach plant carrying the alpha-WOLF 27 allele and further comprising a genetic determinant resulting in resistance against Peronospora effusa races Pe:1 to Pe:19. The genetic determinant can be another resistance conferring alpha/beta-WOLF allele or a resistance conferring allele of a totally different gene.

The invention further relates to propagation material comprising the alpha-WOLF 27 allele. In one embodiment, the propagation material is suitable for sexual reproduction. Such propagation material comprises for example a microspore, pollen, ovary, ovule, embryo sac and egg cell. In another embodiment, the propagation material is suitable for vegetative reproduction. Such propagation material comprises for example a cutting, root, stem, cell, protoplast, and a tissue culture of regenerable cells. A part of the plant that is suitable for preparing tissue cultures is in particular a leaf, pollen, an embryo, a cotyledon, a hypocotyl, a meristematic cell, a root tip, an anther, a flower, a seed and a stem.

The invention furthermore relates to a cell of a spinach plant comprising the alpha-WOLF 27 allele. Such a cell may be either in isolated form or may be part of the complete plant or parts thereof and then still constitutes a cell of the invention because such a cell harbors the alpha-WOLF 27 allele that confers resistance to downy mildew. Each cell of a plant of the invention carries the genetic information that confers resistance to Peronospora effusa. Such a cell of the invention may also be a regenerable cell that may be used to regenerate a new plant comprising the allele of the invention.

Yet another aspect of the invention relates to a method for making a hybrid spinach seed comprising crossing a first parent spinach plant with a second parent spinach plant and harvesting the resultant hybrid spinach seed, wherein said first and/or second parent spinach plant comprises the alpha-WOLF 27 allele. In particular embodiment, the first and/or second parent plant is a plant of an inbred line as defined herein.

The invention further relates to a hybrid spinach plant grown from seed produced by crossing a first parent spinach plant with a second parent spinach plant and harvesting the resultant hybrid spinach seed, wherein said first and/or second parent spinach plant comprises the alpha-WOLF 27 allele.

Determining the genomic DNA or coding DNA sequence of at least part of a WOLF gene in the genome of a spinach plant may be performed using any suitable molecular biological method known in the art, including but not limited to (genomic) PCR amplification followed by Sanger sequencing, whole-genome-sequencing, transcriptome sequencing, sequence-specific target capture followed by next-generation sequencing (using, for example, the xGen© target capture system of Integrated DNA Technologies), specific amplification of LRR-domain-comprising gene sequences (using, for example, the RenSeq methodology, as described in U.S. patent application Ser. No. 14/627,116, and in Jupe et al., 2013, Plant J. 76: 530-544) followed by sequencing, etcetera.

In one embodiment the invention relates to a method for identifying a plant carrying the alpha-WOLF 27 allele comprises determining the DNA sequence coding for the LRR domain as defined herein.

In a further embodiment of the method the LRR domain of the alpha-WOLF 27 allele is determined by using a primer pair to amplify the genomic DNA region of the LRR domain. The forward primer is preferably a nucleic acid molecule having the sequence of SEQ ID NO: 4 and the reverse primer is preferably a nucleic acid molecule having the sequence of SEQ ID NO: 5.

Another aspect of the invention relates to a method for producing a spinach plant comprising resistance to Peronospora effusa comprising: (a) crossing a plant comprising the alpha-WOLF 27 allele, with another plant; (b) optionally performing one or more rounds of selfing and/or crossing; (c) optionally selecting after each round of selfing or crossing for a plant that comprises the alpha-WOLF 27 allele.

Selecting a plant comprising the alpha-WOLF 27 allele can be done by determining the presence of the DNA sequence of the NBS-LRR domain of the allele having in order of increased preference 97%, 97.3%, 97.5%, 97.8%, 98%, 98.3%, 98.5%, 98.8%, 99%, 99.1%, 99.2%, 99.3%, 99.4%, 99.5%, 99.6%, 99.7%, 99.8%, 99.9%, 100% sequence identity to SEQ ID NO: 9.

In another embodiment, selecting a plant comprising the alpha-WOLF 27 allele can be done by determining the presence the coding sequence of the entire allele.

Alternatively, the presence of the alpha-WOLF 27 allele can be determined phenotypically by assaying a plant in a disease test, for example the test as described herein.

The invention further relates to the use of a spinach plant carrying the alpha-WOLF 27 allele in breeding to confer resistance against Peronospora effusa.

The invention also relates to a breeding method for the development of spinach plants carrying the alpha-WOLF 27 allele of the invention wherein germplasm which comprises said allele is used. Seed capable of growing into a plant comprising the allele of the invention and being representative for the germplasm was deposited with the NCIMB under accession number NCIMB 43668.

In another aspect, the invention relates to a method for the production of a spinach plant which comprises alpha-WOLF 27 allele, which method comprises: (a) crossing a plant comprising the allele with another plant; (b) optionally selecting for plants comprising said allele in the F1; (c) optionally backcrossing the resulting F1 with the preferred parent and selecting for plants that have the said allele in the BC1F1; (d) optionally performing one or more additional rounds of selfing, crossing, and/or backcrossing, and subsequently selecting for a plant which comprises the said allele or shows the resistance profile corresponding to said allele. The invention also encompasses a spinach plant produced by this method.

The invention also relates to a harvested leaf of a spinach plant of the invention, to a food product which comprises a harvested leaf of a spinach plant of the invention, either in natural or in processed form.

Spinach leaves are sold in packaged form, including without limitation as pre-packaged spinach leaves or as processed in a salad comprising said leaves. Mention of such a package is e.g. made in U.S. Pat. No. 5,523,136, which provides packaging film, and packages from such packaging film, including such packaging containing leafy produce, and methods for making and using such packaging film and packages, which are suitable for use with the spinach leaves of the invention. Thus, the invention comprehends the use of and methods for making and using the leaves of the spinach plant of the invention, as well as leaves of spinach plants derived from the invention.

The invention further relates to a container which comprises one or more plants of the invention, or one or more spinach plants derived from a plant of the invention, in a growth substrate for harvest of leaves from the plant, in a domestic environment. This way the consumer may pick very fresh leaves for use in salads, when the plant is in a ready-to-harvest condition.

The invention also relates to the use of a spinach plant, of which representative seed was deposited with the NCIMB under accession number NCIMB 43668, in the production of a spinach plant comprising the alpha-WOLF 27 allele.

In a further embodiment the said spinach plant is a hybrid, doubled haploid, or inbred spinach plant.

The spinach plant of the invention may comprise the alpha-WOLF 27 allele heterozygously or homozygously.

Another aspect of the invention is the use of a cell comprising the alpha-WOLF 27 allele for the production of a spinach plant showing resistance to Peronospora effusa.

The invention relates to an allele designated alpha-WOLF 27 which when present in a spinach plant homozygously confers complete resistance to at least Peronospora effusa race Pe: 7, Pe: 8, Pe: 9, Pe: 11, Pe: 12, Pe: 13, Pe: 14, Pe: 15, Pe: 17. In particular, the alpha-WOLF 27 allele when present in a spinach plant homozygously confers complete resistance to at least Peronospora effusa race Pe: 1, Pe: 2, Pe: 3, Pe: 4, Pe: 5, Pe: 6, Pe: 7, Pe: 8, Pe: 9, Pe: 11, Pe: 12, Pe: 13, Pe: 14, Pe: 15, Pe: 17. More in particular, the alpha-WOLF 27 allele when present in a spinach plant homozygously confers complete resistance to at least Peronospora effusa race Pe: 1, Pe: 2, Pe: 3, Pe: 4, Pe: 5, Pe: 6, Pe: 7, Pe: 8, Pe: 9, Pe: 11, Pe: 12, Pe: 13, Pe: 14, Pe: 15, Pe:16, Pe: 17 and Pe:18 and intermediate resistance to Peronospora effusa race Pe:10. In all three cases, the protein encoded by said allele is a CC-NBS-LRR protein that comprises in its amino acid sequence: a) the motif “MAEIGYSVC” (SEQ ID NO: 1) at its N-terminus; and b) the motif “KWMCLR” (SEQ ID NO: 2); and wherein the LRR domain of the protein has in order of increased preference at least 95%, 95.5%, 96%, 96.5%, 97%, 97.5%, 98%, 98.5%, 99%, 99.5%, 100% sequence identity to SEQ ID NO: 10.

The invention further relates to an allele designated alpha-WOLF 27 which when present in a spinach plant homozygously confers complete resistance to at least Peronospora effusa race Pe: 7, Pe: 8, Pe: 9, Pe: 11, Pe: 12, Pe: 13, Pe: 14, Pe: 15 and Pe: 17; or confers complete resistance to at least Peronospora effusa race Pe: 1, Pe: 2, Pe: 3, Pe: 4, Pe: 5, Pe: 6, Pe: 7, Pe: 8, Pe: 9, Pe: 11, Pe: 12, Pe: 13, Pe: 14, Pe: 15 and Pe: 17; or confers complete resistance to at least Peronospora effusa race Pe: 1, Pe: 2, Pe: 3, Pe: 4, Pe: 5, Pe: 6, Pe: 7, Pe: 8, Pe: 9, Pe: 11, Pe: 12, Pe: 13, Pe: 14, Pe: 15, Pe:16, Pe: 17 and Pe:18 and intermediate resistance to Peronospora effusa race Pe:10, wherein the protein encoded by said allele is a CC-NBS-LRR protein that comprises in its amino acid sequence: a) the motif “MAEIGYSVC” (SEQ ID NO: 1) at its N-terminus; and b) the motif “KWMCLR” (SEQ ID NO: 2); and wherein the LRR domain of the protein has in order of increased preference at least 99.5% sequence identity to SEQ ID NO: 10.

The invention further relates to an allele designated alpha-WOLF 27 which when present in a spinach plant homozygously confers complete resistance to at least Peronospora effusa race Pe: 7, Pe: 8, Pe: 9, Pe: 11, Pe: 12, Pe: 13, Pe: 14, Pe: 15 and Pe: 17; or confers complete resistance to at least Peronospora effusa race Pe: 1, Pe: 2, Pe: 3, Pe: 4, Pe: 5, Pe: 6, Pe: 7, Pe: 8, Pe: 9, Pe: 11, Pe: 12, Pe: 13, Pe: 14, Pe: 15 and Pe: 17; or confers complete resistance to at least Peronospora effusa race Pe: 1, Pe: 2, Pe: 3, Pe: 4, Pe: 5, Pe: 6, Pe: 7, Pe: 8, Pe: 9, Pe: 11, Pe: 12, Pe: 13, Pe: 14, Pe: 15, Pe:16, Pe: 17 and Pe:18 and intermediate resistance to Peronospora effusa race Pe:10, wherein the protein encoded by said allele is a CC-NBS-LRR protein that comprises in its amino acid sequence: a) the motif “MAEIGYSVC” (SEQ ID NO: 1) at its N-terminus; and b) the motif “KWMCLR” (SEQ ID NO: 2); and wherein the LRR domain of the protein has in order of increased preference at least 95%, 95.5%, 96%, 96.5%, 97%, 97.5%, 98%, 98.5%, 99%, 99.5%, 100% sequence identity to SEQ ID NO: 10, and wherein the DNA sequence of the LRR domain in order of increased preference has at least 97%, 97.3%, 97.5%, 97.8%, 98%, 98.3%, 98.5%, 98.8%, 99%, 99.3%, 99.5%, 99.8%, 100% sequence identity to SEQ ID NO: 9.

The invention further relates to an allele designated alpha-WOLF 27 which when present in a spinach plant homozygously confers complete resistance to at least Peronospora effusa race Pe: 7, Pe: 8, Pe: 9, Pe: 11, Pe: 12, Pe: 13, Pe: 14, Pe: 15 and Pe: 17; or confers complete resistance to at least Peronospora effusa race Pe: 1, Pe: 2, Pe: 3, Pe: 4, Pe: 5, Pe: 6, Pe: 7, Pe: 8, Pe: 9, Pe: 11, Pe: 12, Pe: 13, Pe: 14, Pe: 15 and Pe: 17; or confers complete resistance to at least Peronospora effusa race Pe: 1, Pe: 2, Pe: 3, Pe: 4, Pe: 5, Pe: 6, Pe: 7, Pe: 8, Pe: 9, Pe: 11, Pe: 12, Pe: 13, Pe: 14, Pe: 15, Pe:16, Pe: 17 and Pe:18 and intermediate resistance to Peronospora effusa race Pe:10, wherein the protein encoded by said allele is a CC-NBS-LRR protein that comprises in its amino acid sequence: a) the motif “MAEIGYSVC” (SEQ ID NO: 1) at its N-terminus; and b) the motif “KWMCLR” (SEQ ID NO: 2); and wherein the DNA sequence of the LRR domain in order of increased preference has at least 97%, 97.3%, 97.5%, 97.8%, 98%, 98.3%, 98.5%, 98.8%, 99%, 99.3%, 99.5%, 99.8%, 100% sequence identity to SEQ ID NO: 9.

The invention also relates to a spinach plant comprising an allele designated alpha-WOLF 27 which when present in a spinach plant homozygously confers complete resistance to at least Peronospora effusa race Pe: 7, Pe: 8, Pe: 9, Pe: 11, Pe: 12, Pe: 13, Pe: 14, Pe: 15, Pe: 17. In particular, the alpha-WOLF 27 allele when present in a spinach plant homozygously confers complete resistance to at least Peronospora effusa race Pe: 1, Pe: 2, Pe: 3, Pe: 4, Pe: 5, Pe: 6, Pe: 7, Pe: 8, Pe: 9, Pe: 11, Pe: 12, Pe: 13, Pe: 14, Pe: 15, Pe: 17. More in particular, the alpha-WOLF 27 allele when present in a spinach plant homozygously confers complete resistance to at least Peronospora effusa race Pe: 1, Pe: 2, Pe: 3, Pe: 4, Pe: 5, Pe: 6, Pe: 7, Pe: 8, Pe: 9, Pe: 11, Pe: 12, Pe: 13, Pe: 14, Pe: 15, Pe:16, Pe: 17 and Pe:18 and intermediate resistance to Peronospora effusa race Pe:10. In all three cases, the protein encoded by said allele is a CC-NBS-LRR protein that comprises in its amino acid sequence: a) the motif “MAEIGYSVC” (SEQ ID NO: 1) at its N-terminus; and b) the motif “KWMCLR” (SEQ ID NO: 2); and wherein the LRR domain of the protein has in order of increased preference at least 95%, 95.3%, 95.5%, 95.8%, 96%, 96.3%, 96.5%, 96.8%, 97%, 97.3%, 97.5%, 97.8%, 98%, 98.3%, 98.5%, 98.8%, 99%, 99.3%, 99.5% 99.8%, 100% sequence identity to SEQ ID NO: 10. Preferably this spinach plant is an agronomically elite spinach plant.

The invention also relates to a spinach plant comprising an allele designated alpha-WOLF 27 which when present in a spinach plant homozygously confers complete resistance to at least Peronospora effusa race Pe: 7, Pe: 8, Pe: 9, Pe: 11, Pe: 12, Pe: 13, Pe: 14, Pe: 15 and Pe: 17; or confers complete resistance to at least Peronospora effusa race Pe: 1, Pe: 2, Pe: 3, Pe: 4, Pe: 5, Pe: 6, Pe: 7, Pe: 8, Pe: 9, Pe: 11, Pe: 12, Pe: 13, Pe: 14, Pe: 15 and Pe: 17; or confers complete resistance to at least Peronospora effusa race Pe: 1, Pe: 2, Pe: 3, Pe: 4, Pe: 5, Pe: 6, Pe: 7, Pe: 8, Pe: 9, Pe: 11, Pe: 12, Pe: 13, Pe: 14, Pe: 15, Pe:16, Pe: 17 and Pe:18 and intermediate resistance to Peronospora effusa race Pe:10, wherein the protein encoded by said allele is a CC-NBS-LRR protein that comprises in its amino acid sequence: a) the motif “MAEIGYSVC” (SEQ ID NO: 1) at its N-terminus; and b) the motif “KWMCLR” (SEQ ID NO: 2); and wherein the LRR domain of the protein has in order of increased preference at least 99.8% sequence identity to SEQ ID NO: 10. Preferably this spinach plant is an agronomically elite spinach plant.

The invention also relates to a spinach plant comprising an allele designated alpha-WOLF 27 which when present in a spinach plant homozygously confers complete resistance to at least Peronospora effusa race Pe: 7, Pe: 8, Pe: 9, Pe: 11, Pe: 12, Pe: 13, Pe: 14, Pe: 15 and Pe: 17; or confers complete resistance to at least Peronospora effusa race Pe: 1, Pe: 2, Pe: 3, Pe: 4, Pe: 5, Pe: 6, Pe: 7, Pe: 8, Pe: 9, Pe: 11, Pe: 12, Pe: 13, Pe: 14, Pe: 15 and Pe: 17; or confers complete resistance to at least Peronospora effusa race Pe: 1, Pe: 2, Pe: 3, Pe: 4, Pe: 5, Pe: 6, Pe: 7, Pe: 8, Pe: 9, Pe: 11, Pe: 12, Pe: 13, Pe: 14, Pe: 15, Pe:16, Pe: 17 and Pe:18 and intermediate resistance to Peronospora effusa race Pe:10, wherein the protein encoded by said allele is a CC-NBS-LRR protein that comprises in its amino acid sequence: a) the motif “MAEIGYSVC” (SEQ ID NO: 1) at its N-terminus; and b) the motif “KWMCLR” (SEQ ID NO: 2); and wherein the LRR domain of the protein has in order of increased preference at least 95%, 95.3%, 95.5%, 95.8%, 96%, 96.3%, 96.5%, 96.8%, 97%, 97.3%, 97.5%, 97.8%, 98%, 98.3%, 98.5%, 98.8%, 99%, 99.3%, 99.5% 99.8%, 100% sequence identity to SEQ ID NO: 10, and wherein the DNA sequence of the LRR domain in order of increased preference has at least 97%, 97.3%, 97.5%, 97.8%, 98%, 98.3%, 98.5%, 98.8%, 99%, 99.3%, 99.5%, 99.8%, 100% sequence identity to SEQ ID NO: 9. Preferably this spinach plant is an agronomically elite spinach plant.

The invention also relates to a spinach plant comprising an allele designated alpha-WOLF 27 which when present in a spinach plant homozygously confers complete resistance to at least Peronospora effusa race Pe: 7, Pe: 8, Pe: 9, Pe: 11, Pe: 12, Pe: 13, Pe: 14, Pe: 15 and Pe: 17; or confers complete resistance to at least Peronospora effusa race Pe: 1, Pe: 2, Pe: 3, Pe: 4, Pe: 5, Pe: 6, Pe: 7, Pe: 8, Pe: 9, Pe: 11, Pe: 12, Pe: 13, Pe: 14, Pe: 15 and Pe: 17; or confers complete resistance to at least Peronospora effusa race Pe: 1, Pe: 2, Pe: 3, Pe: 4, Pe: 5, Pe: 6, Pe: 7, Pe: 8, Pe: 9, Pe: 11, Pe: 12, Pe: 13, Pe: 14, Pe: 15, Pe:16, Pe: 17 and Pe:18 and intermediate resistance to Peronospora effusa race Pe:10, wherein the protein encoded by said allele is a CC-NBS-LRR protein that comprises in its amino acid sequence: a) the motif “MAEIGYSVC” (SEQ ID NO: 1) at its N-terminus; and b) the motif “KWMCLR” (SEQ ID NO: 2); and wherein the DNA sequence of the LRR domain in order of increased preference has at least 97%, 97.3%, 97.5%, 97.8%, 98%, 98.3%, 98.5%, 98.8%, 99%, 99.3%, 99.5%, 99.8%, 100% sequence identity to SEQ ID NO: 9. Preferably this spinach plant is an agronomically elite spinach plant.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### Examples

### Example 1: Testing for Resistance to Peronspora effusa in Spinach Plants

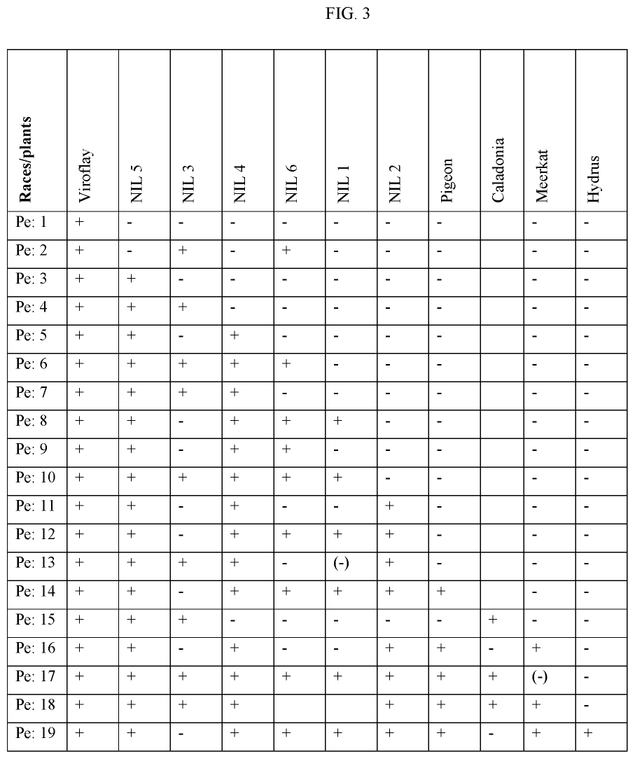

The resistance to downy mildew infection was assayed as described by Irish et al. (2008; Phytopathol. 98: 894-900), using a differential set. Spinach plants of the invention were sown along with spinach plants from different other genotypes (see FIG. 3) in trays containing Scotts Redi-Earth medium, and fertilized twice a week after seedling emergence with Osmocote Peter's (13-13-13) fertilizer (Scotts). Plants were inoculated with a sporangial suspension (2.5×10⁵/ml) of a pathogenic race of Peronospora effusa at the first true leaf stage. In this manner, 4 officially recognized pathogenic race were tested.

The inoculated plants were placed in a dew chamber at 18° C. with 100% relative humidity for a 24 h period, and then moved to a growth chamber at 18° C. with a 12 h photoperiod for 6 days. After 6 days, the plants were returned to the dew chamber for 24 h to induce sporulation, and they were scored for disease reaction.

Plants for this specific test were scored as resistant, intermediately resistant, or susceptible based on symptoms of chlorosis and signs of pathogen sporulation on the cotyledons and true leaves, as described by Irish et al. (2007; Plant Dis. 91: 1392-1396). Plants exhibiting no evidence of chlorosis and sporulation were in this specific test considered as resistant. Resistant plants were re-inoculated to assess whether plants initially scored as resistant had escaped infection, or whether they were truly resistant. Plants that showed only symptoms of chlorosis, or sporulation occurring only on the tips of the cotyledons were scored as intermediately resistant. Plants showing more than these symptoms of downy mildew infection were scored as being susceptible.

FIG. 1 shows the resistance of a plant carrying the alpha-WOLF 27 allele to each one of these pathogenic races. FIG. 3 shows the differential set of spinach downy mildew races and the resistance of various spinach varieties (hybrids) to each one of these pathogenic races. A susceptible reaction is scored as “+” (indicating a successful infection by the fungus, with sporulation occurring on the entire cotyledon), and resistance is depicted as “−” (absence of sporulation on the cotyledons). An intermediate resistance response is indicated as “(−)”, which in practice means a slightly level of infection (with only symptoms of chlorosis, or sporulation only occurring on the tips of the cotyledons in the differential seedling test).

### Example 2: Amplification of the LRR Domain-Encoding Region

The isolated genomic DNA of a spinach plant comprising the alpha-WOLF 27 allele, of which a representative sample of seed was deposited with the NCIMB under accession number NCIMB 43668 was used in polymerase chain reactions (PCR), using forward primer ACAAGTGGATGTGTCTTAGG (SEQ ID NO: 4) and reverse primer TTCGCCCTCATCTTCCTGG (SEQ ID NO: 5). The primer pair amplifies the LRR domain-encoding region of an alpha-WOLF gene, and has been designed for selectively amplifying part of a WOLF gene, and not of other CC-NBS-LRR protein-encoding genes.

PCR conditions for amplifying the LRR domain-encoding region of an alpha-WOLF gene using primers having SEQ ID NO: 4 and SEQ ID NO: 5 were as follows, using Platinum Taq enzyme (Thermo Fisher Scientific):
3 minutes at 95° C. (initial denaturing step)
40 amplification cycles, each cycle consisting of: 30 seconds denaturation at 95° C., 30 seconds annealing at 60° C., and 30 seconds extension at 72° C.
2 minutes at 72° C. (final extension step)

The isolated genomic DNA of a spinach plant of variety Viroflay comprising the beta-WOLF 0 allele was used in polymerase chain reactions (PCR), using forward primer TCACGTGGGTTGTGTTGT (SEQ ID NO: 6) and reverse primer TTCGCCCTCATCTTCCTGG (SEQ ID NO: 5). The primer pair amplifies the LRR domain-encoding region of a beta-WOLF gene, and has been designed for selectively amplifying part of a WOLF gene, and not of other CC-NBS-LRR protein-encoding genes.

PCR conditions for amplifying the LRR domain-encoding region of a beta-WOLF gene using primers having SEQ ID NO: 5 and SEQ ID NO: 6 were as follows, using Platinum Taq enzyme (Thermo Fisher Scientific):
3 minutes at 95° C. (initial denaturing step)
40 amplification cycles, each cycle consisting of: 30 seconds denaturation at 95° C., 50 seconds annealing at 58° C. and 50 seconds extension at 72° C.
2 minutes at 72° C. (final extension step)

The PCR products were visualized on agarose gel (not shown), and DNA was purified from the PCR reaction. Subsequently the sequence of the PCR products was determined using methods well known in the art.

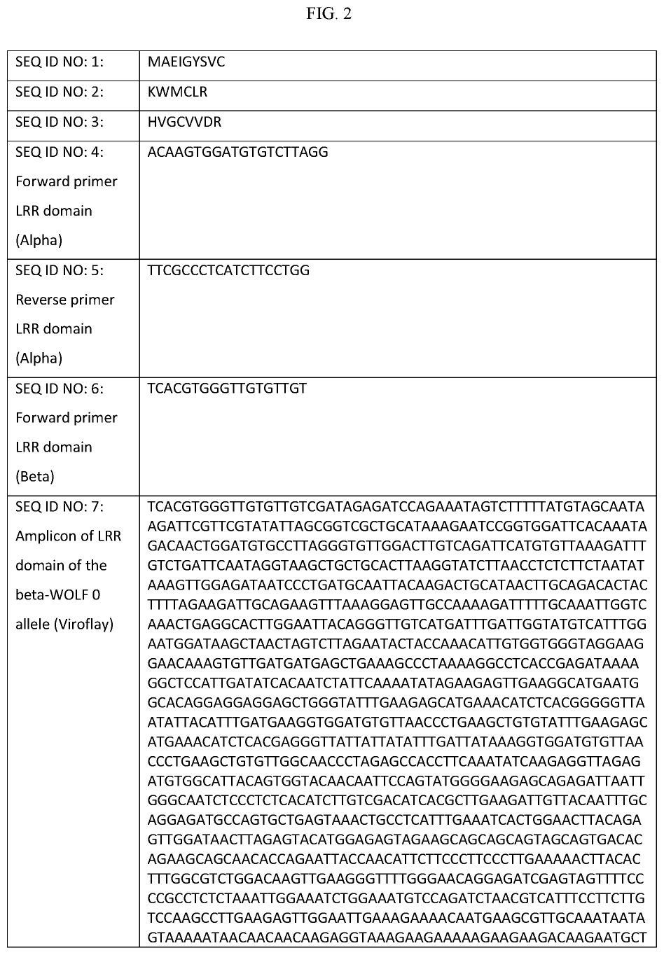

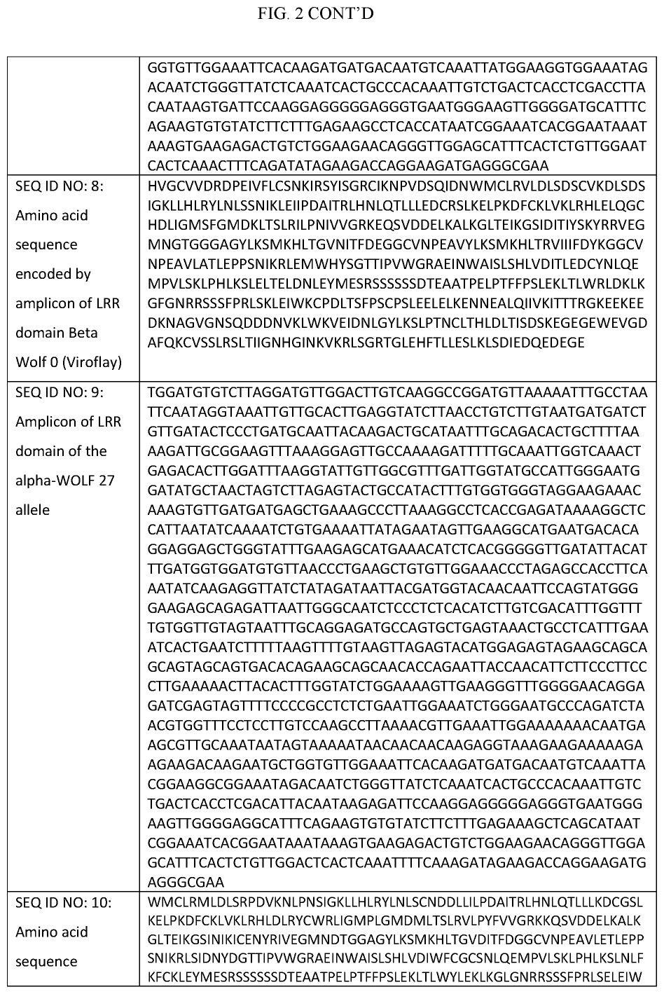

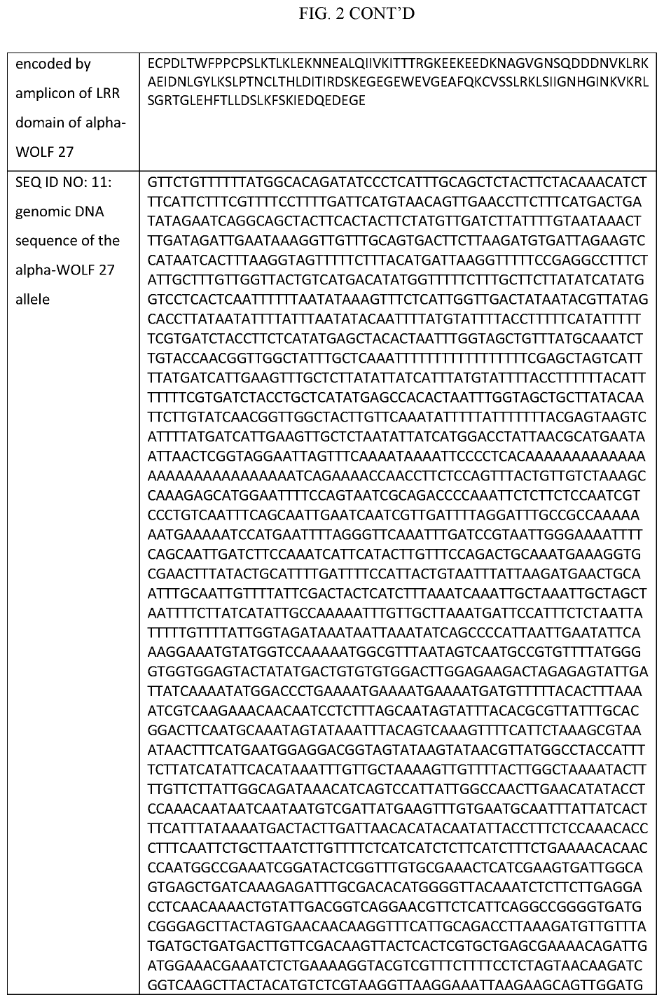

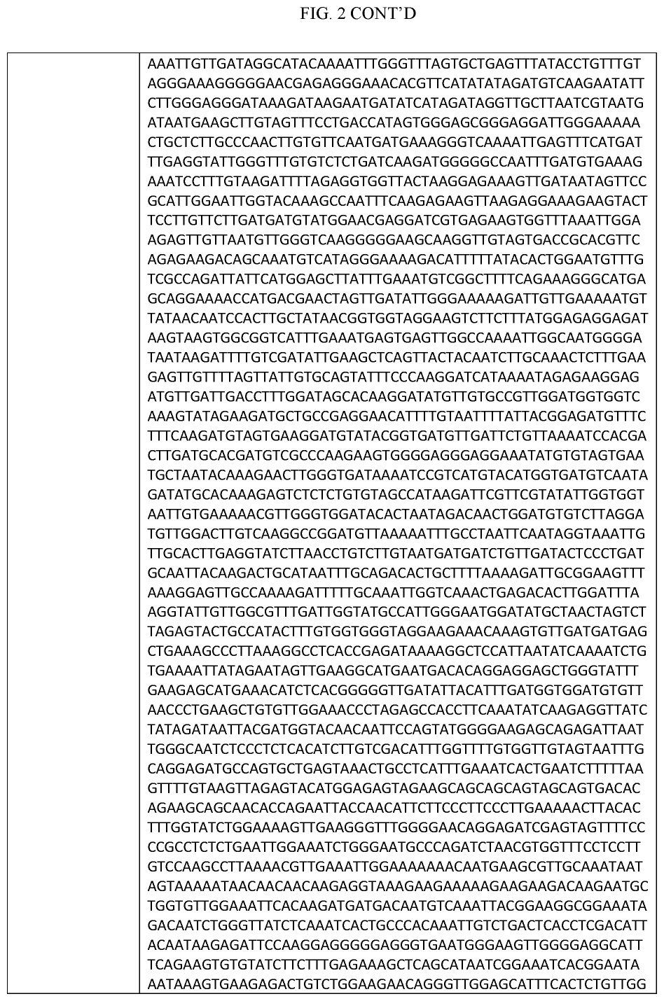

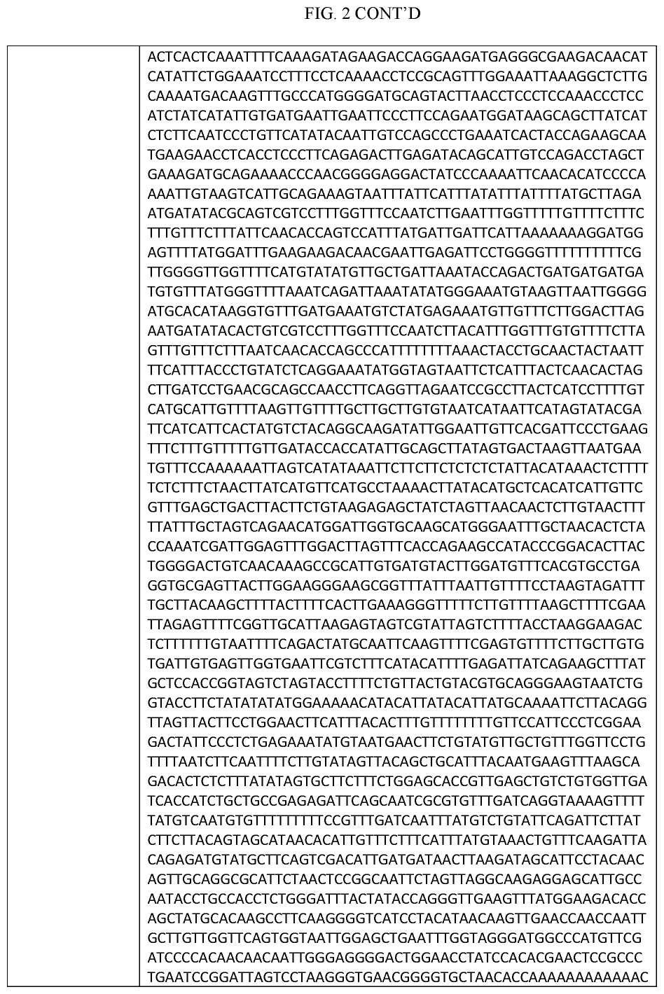

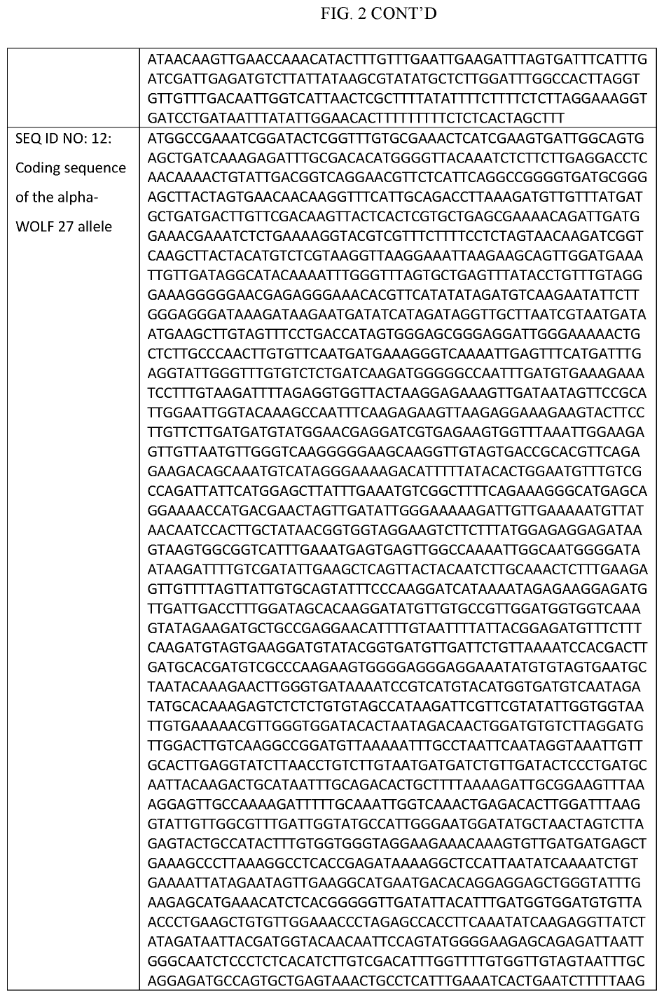

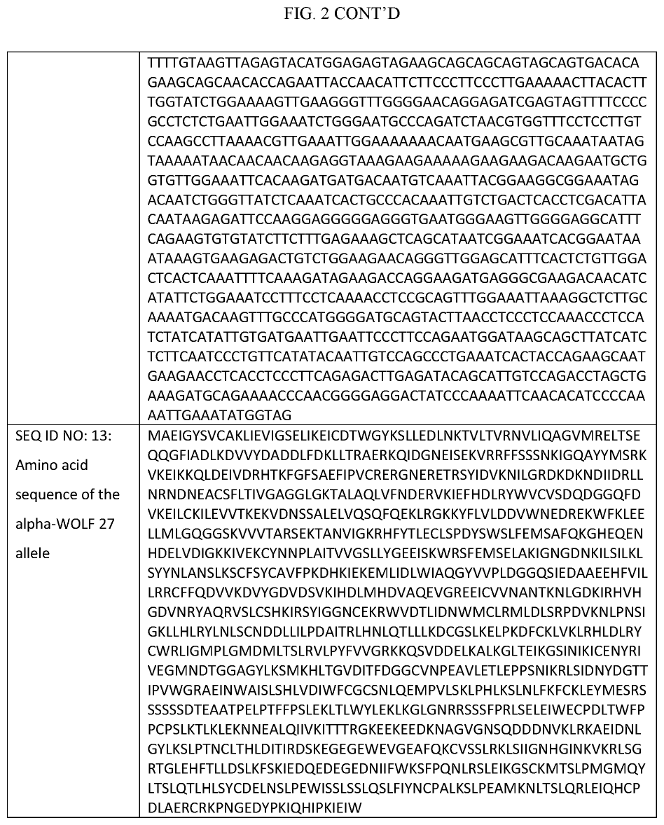

The DNA sequence of the LRR domain of the alpha-WOLF 27 allele amplified by primers having SEQ ID NO: 4 and SEQ ID NO: 5 is provided in FIGS. 2A-2H under SEQ ID NO: 9.

The DNA sequence of the LRR domain of the beta-WOLF 0 allele amplified by primers having SEQ ID NO: 5 and SEQ ID NO: 6 is provided in FIGS. 2A-2H under SEQ ID NO: 7.

Finally, the obtained sequences were translated into the corresponding amino acid sequence of the LRR domain having SEQ ID NO: 10 and SEQ ID NO: 8 for the alpha-WOLF 27 allele and the beta-WOLF 0, respectively (See also FIGS. 2A-2H).

If PCR products were to be sequenced using SMRT sequencing (Pacific Biosciences), PCR primers and PCR conditions were different.

To the above-mentioned forward primers the following standard amplification sequence was added: GCAGTCGAACATGTAGCTGACTCAGGTCAC.

To the reverse primer, the following standard amplification sequence was added: TGGATCACTTGTGCAAGCATCACATCGTAG.

### Example 3: Introducing an Alpha-WOLF 27 Allele in a Plant not Carrying the Allele

A spinach plant comprising the alpha-WOLF 27 allele, of which a representative sample of seed was deposited with the NCIMB under accession number NCIMB 43668 was crossed with a plant of variety Viroflay carrying the beta-WOLF 0 allele to obtain a F1 generation. Subsequently, a F1 plant was selfed to obtain a F2 population.

Plants of the F2 population were assayed as described in Example 1 for resistance to Peronospora effusa Pe: 14, Pe: 15 and Pe: 17.

Genomic DNA of each plant of the same F2 population was isolated and used in two different polymerase chain reactions (PCR). The first PCR reaction was done using primers for amplifying the LRR domain of an alpha-WOLF allele and the second PCR reaction was done using primers for amplifying the LRR domain of a beta-WOLF allele, both as described in Example 2.

The PCR products were visualized on agarose gel (not shown), this demonstrated that approximately 75% of the plants contained an alpha-WOLF fragment, and that the remaining approximately 25% of the plants only contained a beta-WOLF fragment. The plants only comprising the beta-WOLF fragment completely correlated with the plants that scored susceptible for Pe: 14, Pe: 15 and Pe: 17.

DNA from the PCR reaction was purified, and subsequently the sequence of the PCR products was determined. The alpha-WOLF PCR products gave a sequence that corresponded to the sequence of SEQ ID NO: 9, the genomic sequence of the LRR domain of the alpha-WOLF 27 allele. The beta-WOLF PCR products gave a sequence that corresponded to the sequence of SEQ ID NO: 7 the genomic sequence of the LRR domain of the beta-WOLF 0 allele.

The invention is further described by the following numbered paragraphs:
1. An agronomically elite spinach plant comprising an allele which confers resistance to at least one Peronospora effusa race when present in a spinach plant and encodes a protein that in order of increased preference has at least 96.5%, 96.8%, 97%, 97.3%, 97.5%, 97.8%, 98%, 98.3%, 98.5%, 98.8%, 99%, 99.3%, 99.5%, 99.8%, 100% sequence identity to a protein comprising an amino acid sequence SEQ ID NO: 13; wherein said protein comprises in its amino acid sequence: a) SEQ ID NO: 1, b) SEQ ID NO: 2, and wherein the LRR domain of the protein has in order of increased preference at least 95%, 95.3%, 95.5%, 95.8%, 96%, 96.3%, 96.5%, 96.8%, 97%, 97.3%, 97.5%, 97.8%, 98%, 98.3%, 98.5%, 98.8%, 99%, 99.3%, 99.5% 99.8%, 100% sequence identity to SEQ ID NO: 10.
2. The agronomically elite spinach plant of paragraph 1, wherein the allele when homozygously present in a spinach plant encodes a protein that confers complete resistance to at least Peronospora effusa races Pe: 7, Pe: 8, Pe: 9, Pe: 11, Pe: 12, Pe: 13, Pe: 14, Pe: 15, Pe: 17.
3. The agronomically elite spinach plant of paragraph 1, wherein the allele when homozygously present in a spinach plant encodes a protein that confers complete resistance to at least Peronospora effusa races Pe: 1, Pe: 2, Pe: 3, Pe: 4, Pe: 5, Pe: 6, Pe: 7, Pe: 8, Pe: 9, Pe: 11, Pe: 12,Pe: 13,Pe: 14,Pe: 15,Pe: 17.
4. The agronomically elite spinach plant of paragraph 1, wherein the allele when homozygously present in a spinach plant encodes a protein that confers complete resistance to at least Peronospora effusa races Pe: 1, Pe: 2, Pe: 3, Pe: 4, Pe: 5, Pe: 6, Pe: 7, Pe: 8, Pe: 9, Pe: 11, Pe: 12, Pe: 13, Pe: 14, Pe: 15, Pe: 16, Pe: 17, Pe: 18 and confers intermediate resistance to at least Peronospora effusa race Pe: 10.
5. An agronomically elite spinach plant comprising an allele which when homozygously present in a spinach plant encodes a protein that confers complete resistance to at least Peronospora effusa races Pe: 1, Pe: 2, Pe: 3, Pe: 4, Pe: 5, Pe: 6, Pe: 7, Pe: 8, Pe: 9, Pe: 11, Pe: 12, Pe: 13, Pe: 14, Pe: 15, Pe: 17, wherein the allele has a nucleotide sequence which has in order of increased preference at least 95%, 95.3%, 95.5%, 95.8%, 96%, 96.3%, 96.5%, 96.8%, 97%, 97.3%, 97.5% 97.8%, 98%, 98.3%, 98.5%, 98.8%, 99%, 99.3% 99.5% 99.8%, 100% sequence identity to SEQ ID NO: 12.
6. The agronomically elite spinach plant of any of the paragraphs 1 to 5, of which a representative sample of seed capable of growing into a plant comprising said allele was deposited with the NCIMB under accession number NCIMB 43668.
7. The agronomically elite spinach plant of any of the paragraphs 1 to 6, wherein the agronomically elite spinach is a plant of a hybrid variety or a plant of an inbred line.
8. A propagation material capable of developing into the agronomically elite spinach plant of any of the paragraphs 1 to 7 and wherein the propagation material comprises a microspore, a pollen, an ovary, an ovule, an embryo, an embryo sac, an egg cell, a cutting, a root tip, a hypocotyl, a cotyledon, a stem, a leaf, a flower, an anther, a seed, a meristematic cell, a protoplast, a cell, or a tissue culture thereof.
9. A cell of the agronomically elite spinach plant of any of the paragraphs 1 to 7.
10. A method of producing an F1 hybrid spinach seed comprising crossing a first parent spinach plant with a second parent spinach plant and harvesting the resultant hybrid spinach seed, wherein said first parent spinach plant and/or said second parent spinach plant is the agronomically elite spinach plant of any of the paragraphs 1 to 7.
11. The method of paragraph 10, wherein the first and/or the second parent plant is a plant of an inbred line.
12. An F1 hybrid spinach plant grown from the seed produced by the method of paragraph 10 or 11, wherein the F1 hybrid plant carries the allele which confers resistance to at least one Peronospora effusa race when present in a spinach plant and encoding a CC-NBS-LRR protein that in order of increased preference has at least 96.5%, 96.8%, 97%, 97.3%, 97.5%, 97.8%, 98%, 98.3%, 98.5%, 98.8%, 99%, 99.3%, 99.5%, 99.8%, 100% sequence identity to a protein comprising an amino acid sequence SEQ ID NO: 13; wherein said protein comprises in its amino acid sequence: (a) SEQ ID NO: 1, (b) SEQ ID NO: 2, and wherein the LRR domain of the protein has in order of increased preference at least 95%, 95.3%, 95.5%, 95.8%, 96%, 96.3%, 96.5%, 96.8%, 97%, 97.3%, 97.5%, 97.8%, 98%, 98.3%, 98.5%, 98.8%, 99%, 99.3%, 99.5%, 99.8%, 100% sequence identity to SEQ ID NO: 10.
13. A method for producing a spinach plant showing resistance to Peronospora effusa comprising: (a) crossing the agronomically elite spinach plant of any of the paragraphs 1 to 7 with another spinach plant; (b) optionally performing one or more rounds of selfing and/or crossing; (c) optionally selecting after the crossing or the one or more rounds of selfing and/or crossing for a plant that comprises said allele.
14. The method of paragraph 13, wherein the method includes performing the optional selection, and the selection of the plant comprising the allele expressing the protein comprises determining the presence of the allele according to a method comprising any or more of; determining the presence of a genomic nucleotide sequence in the genome of a plant, wherein said sequence in order of increased preference at least 90%, 90.5%, 91%, 91.5%, 92%, 92.5%, 93%, 93.5%, 94%, 95%, 95.5%, 96%, 96.5%, 97%, 97.5% 98%, 98.5%, 99%, 99.5% 100% sequence identity to SEQ ID NO: 11, or determining the presence of a nucleotide sequence in a plant, wherein said sequence has in order of increased preference at least 97.8%, 98%, 98.3%, 98.5%, 98.8%, 99%, 99.3%, 99.5%, 99.8%, 100% sequence identity to SEQ ID NO: 12, or determining the presence of a LRR domain as having in order of increased preference at least 97%, 97.3% 97.5% 97.8%, 98%, 98.3%, 98.5%, 98.8%, 99%, 99.3% 99.5% 99.8%, 100% sequence identity to SEQ ID NO: 9.
15. The method of paragraph 13 or 14, wherein the method includes performing the optional one or more rounds of selfing and/or crossing and the optional selection, and the selection of the plant comprising the allele expressing the protein comprises determining the presence of the allele according to a method comprising any or more of: determining the presence of a genomic nucleotide sequence in the genome of a plant, wherein said sequence in order of increased preference at least 90%, 90.5%, 91%, 91.5%, 92%, 92.5%, 93%, 93.5%, 94%, 95%, 95.5%, 96%, 96.5%, 97%, 97.5%, 98%, 98.5%, 99%, 99.5%, 100% sequence identity to SEQ ID NO: 11, or determining the presence of a nucleotide sequence in a plant, wherein said sequence has in order of increased preference at least 97.8%, 98%, 98.3%, 98.5%, 98.8%, 99%, 99.3%, 99.5%, 99.8%, 100% sequence identity to SEQ ID NO: 12, or determining the presence of a LRR domain as having in order of increased preference at least 97%, 97.3%, 97.5%, 97.8%, 98%, 98.3%, 98.5%, 98.8%, 99%, 99.3%, 99.5%, 99.8%, 100% sequence identity to SEQ ID NO: 9.
16. A method of producing an F1 hybrid spinach seed comprising crossing a first parent spinach plant with a second parent spinach plant and harvesting the resultant hybrid spinach seed, wherein said first parent spinach plant and/or said second parent spinach plant is the agronomically elite spinach plant of any of the paragraphs 1 to 7.
21. An allele designated alpha-WOLF 27 which confers resistance to at least one Peronospora effusa race, wherein the protein encoded by said allele is a CC-NBS-LRR protein that comprises in its amino acid sequence: a) the motif “MAEIGYSVC” (SEQ ID NO: 1) at its N-terminus; and b) the motif “KWMCLR” (SEQ ID NO: 2); and wherein the LRR domain of the protein has in order of increased preference at least 95%, 95.5%, 96%, 96.5%, 97%, 97.5%, 98%, 98.5%, 99%, 99.5%, 100% sequence identity to SEQ ID NO: 10.
22. The allele of paragraph 21 wherein the DNA sequence of the LRR domain in order of increased preference has at least 95%, 95.5%, 96%, 96.5%, 97%, 97.3%, 97.5%, 97.8%, 98%, 98.3%, 98.5%, 98.8%, 99%, 99.3%, 99.5%, 99.8%, 100% sequence identity to SEQ ID NO: 9.
23. The allele of paragraph 21, wherein the allele when homozygously present in a spinach plant confers complete resistance to at least Peronospora effusa race Pe: 7, Pe: 8, Pe: 9, Pe: 11,Pe: 12,Pe: 13,Pe: 14,Pe: 15,Pe: 17.
24. The allele of paragraph 21, wherein the allele when homozygously present in a spinach plant confers complete resistance to at least Peronospora effusa race Pe: 1, Pe: 2, Pe: 3, Pe: 4, Pe: 5, Pe: 6, Pe: 7, Pe: 8, Pe: 9, Pe: 11, Pe: 12, Pe: 13, Pe: 14, Pe: 15, Pe: 17.
25. A spinach plant comprising the allele of paragraph 21, of which a representative sample of seed capable of growing into a plant comprising said allele was deposited with the NCIMB under accession number NCIMB 43668.
26. The spinach plant of paragraph 25, wherein the plant is an agronomically elite plant.
27. The spinach plant of paragraph 26, wherein the agronomically elite plant is a hybrid variety or an inbred line.
28. The spinach plant of paragraph 27, further comprising a genetic determinant resulting in resistance against Peronospora effusa races Pe:1 to Pe:17.
29. A propagation material capable of developing into and/or being derived from the spinach plant of paragraph 4, wherein the propagation material comprises the allele and wherein the propagation material is selected from a group consisting of a microspore, a pollen, an ovary, an ovule, an embryo, an embryo sac, an egg cell, a cutting, a root, a root tip, a hypocotyl, a cotyledon, a stem, a leaf, a flower, an anther, a seed, a meristematic cell, a protoplast, a cell, or a tissue culture thereof.
30. A cell of a spinach plant, which cell comprises the allele of paragraph 1.
31. A method of producing a hybrid spinach seed comprising crossing a first parent spinach plant with a second parent spinach plant and harvesting the resultant hybrid spinach seed, wherein said first parent spinach plant comprises the allele of paragraph 1.
32. The method of paragraph 31, wherein the first and/or second parent is a plant of an inbred line.
33. A hybrid spinach plant grown from the seed produced by the method of paragraph 31.
34. A method for identifying a spinach plant carrying the allele of paragraph 21, comprising determining the presence of the LRR domain as defined in paragraph 21 by determining its nucleotide sequence or a part thereof in a plant, wherein said sequence has in order of increased preference 95%, 95.5%, 96%, 96.5%, 97%, 97.3%, 97.5%, 97.8%, 98%, 98.3%, 98.5%, 98.8%, 99%, 99.3%, 99.5%, 99.8%, 100% sequence identity to SEQ ID NO: 9.
35. The method of paragraph 34, wherein the LRR domain is determined by using a primer pair to amplify the LRR domain, wherein the forward primer is a nucleic acid molecule having the sequence of SEQ ID NO: 4.
36. The method of paragraph 34, wherein the LRR domain is determined by using a primer pair to amplify the LRR domain, wherein the reverse primer is a nucleic acid molecule having the sequence of SEQ ID NO: 5.
37. A method for producing a spinach plant showing resistance to Peronospora effusa comprising:
(a) crossing a plant comprising the allele of paragraph 1, with another plant;
(b) optionally performing one or more rounds of selfing and/or crossing;
(c) selecting after one or more rounds of selfing and/or crossing for a plant that comprises said allele of paragraph 1.
38. The method of paragraph 37, wherein the selection of a plant comprising the allele comprises determining the presence of the allele according the method of paragraph 34.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. An allele designated alpha-WOLF 27 which confers resistance to at least one Peronospora effusa race, wherein the protein encoded by said allele is a CC-NBS-LRR protein that comprises in its amino acid sequence: a) the motif “MAEIGYSVC” (SEQ ID NO: 1) at its N-terminus; and b) the motif “KWMCLR” (SEQ ID NO: 2); and wherein the LRR domain of the protein has in order of increased preference at least 95%, 95.5%, 96%, 96.5%, 97%, 97.5%, 98%, 98.5%, 99%, 99.5%, 100% sequence identity to SEQ ID NO: 10.

2. The allele of claim 1 wherein the DNA sequence of the LRR domain in order of increased preference has at least 95%, 95.5%, 96%, 96.5%, 97%, 97.3%, 97.5%, 97.8%, 98%, 98.3%, 98.5%, 98.8%, 99%, 99.3%, 99.5%, 99.8%, 100% sequence identity to SEQ ID NO: 9.

3. The allele of claim 1, wherein the allele when homozygously present in a spinach plant confers complete resistance to at least Peronospora effusa race Pe: 7, Pe: 8, Pe: 9, Pe: 11, Pe: 12,Pe: 13,Pe: 14,Pe: 15,Pe: 17.

4. The allele of claim 1, wherein the allele when homozygously present in a spinach plant confers complete resistance to at least Peronospora effusa race Pe: 1, Pe: 2, Pe: 3, Pe: 4, Pe:
5, Pe: 6, Pe: 7, Pe: 8, Pe: 9, Pe: 11, Pe: 12, Pe: 13, Pe: 14, Pe: 15, Pe: 17.

5. A spinach plant comprising the allele of claim 1, of which a representative sample of seed capable of growing into a plant comprising said allele was deposited with the NCIMB under accession number NCIMB 43668.

6. The spinach plant of claim 5, wherein the plant is an agronomically elite plant.

7. The spinach plant of claim 6, wherein the agronomically elite plant is a hybrid variety or an inbred line.

8. The spinach plant of claim 7, further comprising a genetic determinant resulting in resistance against Peronospora effusa races Pe:1 to Pe:17.

9. A propagation material capable of developing into and/or being derived from the spinach plant of claim 4, wherein the propagation material comprises the allele and wherein the propagation material is selected from a group consisting of a microspore, a pollen, an ovary, an ovule, an embryo, an embryo sac, an egg cell, a cutting, a root, a root tip, a hypocotyl, a cotyledon, a stem, a leaf, a flower, an anther, a seed, a meristematic cell, a protoplast, a cell, or a tissue culture thereof.

10. A cell of a spinach plant, which cell comprises the allele of claim 1.

11. A method of producing a hybrid spinach seed comprising crossing a first parent spinach plant with a second parent spinach plant and harvesting the resultant hybrid spinach seed, wherein said first parent spinach plant comprises the allele of claim 1.

12. The method of claim 11, wherein the first and/or second parent is a plant of an inbred line.

13. A hybrid spinach plant grown from the seed produced by the method of claim 11.

14. A method for identifying a spinach plant carrying the allele of claim 1, comprising determining the presence of the LRR domain as defined in claim 1 by determining its nucleotide sequence or a part thereof in a plant, wherein said sequence has in order of increased preference 95%, 95.5%, 96%, 96.5%, 97%, 97.3%, 97.5%, 97.8%, 98%, 98.3%, 98.5%, 98.8%, 99%, 99.3%, 99.5%, 99.8%, 100% sequence identity to SEQ ID NO: 9.

15. The method of claim 14, wherein the LRR domain is determined by using a primer pair to amplify the LRR domain, wherein the forward primer is a nucleic acid molecule having the sequence of SEQ ID NO: 4.

16. The method of claim 14, wherein the LRR domain is determined by using a primer pair to amplify the LRR domain, wherein the reverse primer is a nucleic acid molecule having the sequence of SEQ ID NO: 5.

17. A method for producing a spinach plant showing resistance to Peronospora effusa comprising:
(a) crossing a plant comprising the allele of claim 1, with another plant;
(b) optionally performing one or more rounds of selfing and/or crossing;
(c) selecting after one or more rounds of selfing and/or crossing for a plant that comprises said allele of claim 1.

18. The method of claim 17, wherein the selection of a plant comprising the allele comprises determining the presence of the allele according the method of claim 14.
